SEQUENCE LISTING (33 sequences)

SEQ ID NO: 1 - DNA, 40 bp, Homo sapiens
acgtgctggtatacgggaatggaactgttctctgtgacat

SEQ ID NO: 2 - DNA, 40 bp, Homo sapiens
cctttacttctgtgggagtggtgcttcagtctccttggcc

SEQ ID NO: 3 - DNA, 40 bp, Homo sapiens
atgtggaagattacgagcaggcccctgtactagattgact

SEQ ID NO: 4 - DNA, 40 bp, Homo sapiens
acatcattgtgtttgggaaggtagctcgccgccactggct

SEQ ID NO: 5 - DNA, 40 bp, Homo sapiens
gcctggcactgctaggcatgccctcctcccaccttccctt

SEQ ID NO: 6 - DNA, 1128 bp, Homo sapiens
atgaactgcatatccgacttcttcacctatgagaccaccaagtcggtggttgtgaagagctggaccatcgggatcatcaaccgagtagttcagcttctgatcatctcctactttgtagggtgggttttcttgcacgagaaggcttaccaggtacgggacacagccattgagtcctcggtggtaaccaaggtgaagggctccggactctacgccaacagagtcatggatgtgtctgattacgtgacgccacctcagggcacctcggtctttgtcatcatcaccaagatgattgttactgaaaatcagatgcaaggattctgcccagagagtgaggagaaataccgctgtgtatcagacagccagtgcgggcctgagcgcttgccaggtggggggatcctcactggccgctgcgtgaactacagctctgtgctccggacctgtgagatccagggctggtgccccacggaggtggacacagtggaaacgcccatcatgatggaagctgagaacttcactattttcatcaagaacagcatccgtttccccctcttcaactttgagaagggaaacctccttcccaacctgacagccagggacatgaagacctgccgcttccacccggacaaggaccctttctgccccatcttgcgggtaggggacgtggtcaagtttgcggggcaggattttgccaaactggcgcgcacggggggagttctgggcattaagatcggctgggtgtgcgacttggacaaggcctgggaccagtgcatccccaaatactccttcacccggctcgacagcgtttctgagaaaagcagcgtgtccccaggctacaacttcaggtttgccaagtactacaaaatggaaaatggcagtgagtaccgcaccctcctgaaggcttttggcatccgcttcgacgtgctggtatacgggaatggaactgttctctgtgacatcatcctgctcaacttcctcaagggggccgaccagtacaaagccaagaagtttgaggaggtgaatgagactacgctgaaaatcgcggctttgaccaacccagtgtaccccagcgaccagaccacagcggagaagcagtccaccgattcgggggccttctccataggccactag

SEQ ID NO: 7 - protein, 375 aa, Homo sapiens
MNCISDFFTYETTKSVVVKSWTIGIINRVVQLLIISYFVGWVFLHEKAYQVRDTAIESSVVTKVKGSGLYANRVMDVSDYVTPPQGTSVFVIITKMIVTENQMQGFCPESEEKYRCVSDSQCGPERLPGGGILTGRCVNYSSVLRTCEIQGWCPTEVDTVETPIMMEAENFTIFIKNSIRFPLFNFEKGNLLPNLTARDMKTCRFHPDKDPFCPILRVGDVVKFAGQDFAKLARTGGVLGIKIGWVCDLDKAWDQCIPKYSFTRLDSVSEKSSVSPGYNFRFAKYYKMENGSEYRTLLKAFGIRFDVLVYGNGTVLCDIILLNFLKGADQYKAKKFEEVNETTLKIAALTNPVYPSDQTTAEKQSTDSGAFSIGH

SEQ ID NO: 8 - DNA, 1125 bp, Homo sapiens
atgaactgcatatccgacttcttcacctatgagaccaccaagtcggtggttgtgaagagctggaccatcgggatcatcaaccgagtagttcagcttctgatcatctcctactttgtagggtgggttttcttgcacgagaaggcttaccaggtacgggacacagccattgagtcctcggtggtaaccaaggtgaagggctccggactctacgccaacagagtcatggatgtgtctgattacgtgacgccacctcagggcacctcggtctttgtcatcatcaccaagatgattgttactgaaaatcagatgcaaggattctgcccagagagtgaggagaaataccgctgtgtatcagacagccagtgcgggcctgagcgcttgccaggtggggggatcctcactggccgctgcgtgaactacagctctgtgctccggacctgtgagatccagggctggtgccccacggaggtggacacagtggaaacgcccatcatgatggaagctgagaacttcactattttcatcaagaacagcatccgtttccccctcttcaactttgagaagggaaacctccttcccaacctgacagccagggacatgaagacctgccgcttccacccggacaaggaccctttctgccccatcttgcgggtaggggacgtggtcaagtttgcggggcaggattttgccaaactggcgcgcacggggggagttctgggcattaagatcggctgggtgtgcgacttggacaaggcctgggaccagtgcatccccaaatactccttcacccggctcgacagcgtttctgagaaaagcagcgtgtccccaggctacaacttcaggtttgccaagtactacaaaatggaaaatggcagtgagtaccgcaccctcctgaaggcttttggcatccgcttcgacgtgctggtatacgggaatgctggcaagttcaacatcatccccaccatcatcagctctgtggcggcctttacttctgtgggagtggtgcttcagtctccttggccaccaccctgctgcgtcagccctggctggaacgtctccagtcatggagagctcattacctccggaagcagccttcccgttttcaggagactccacctgttatag

SEQ ID NO: 9 - protein, 374 aa, Homo sapiens
MNCISDFFTYETTKSVVVKSWTIGIINRVVQLLIISYFVGWVFLHEKAYQVRDTAIESSVVTKVKGSGLYANRVMDVSDYVTPPQGTSVFVIITKMIVTENQMQGFCPESEEKYRCVSDSQCGPERLPGGGILTGRCVNYSSVLRTCEIQGWCPTEVDTVETPIMMEAENFTIFIKNSIRFPLFNFEKGNLLPNLTARDMKTCRFHPDKDPFCPILRVGDVVKFAGQDFAKLARTGGVLGIKIGWVCDLDKAWDQCIPKYSFTRLDSVSEKSSVSPGYNFRFAKYYKMENGSEYRTLLKAFGIRFDVLVYGNAGKFNIIPTIISSVAAFTSVGVVLQSPWPPPCCVSPGWNVSSHGELITSGSSLPVFRRLHLL

SEQ ID NO: 10 - DNA, 1158 bp, Homo sapiens
atggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcaggcccctgtactagattga

SEQ ID NO: 11 - protein, 385 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQAPVLD

SEQ ID NO: 12 - DNA, 1095 bp, Homo sapiens
atggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggtagctcgccgccactggctcccctccgtcactccctgcagggacaaggggcctctccctgcccctgcagaaacactttttttctttttcggtgtcttggcaggcagggaaatttga

SEQ ID NO: 13 - protein, 364 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKVARRHWLPSVTPCRDKGPLPAPAETLFFFFGVLAGREI

SEQ ID NO: 14 - DNA, 1221 bp, Homo sapiens
atggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatgccctcctcccaccttcccttctccaagaccacccccctcaggtcccagccttctcccaagagatgggagtgcctttccattccggtaaagattccaggcttctcaggaaggggcacgcaaagaataagatgggttgatgggttgcaagcatcctggctcactctcaccctatgctaa

SEQ ID NO: 15 - protein, 406 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMPSSHLPFSKTTPLRSQPSPKRWECLSIPVKIPGFSGRGTQRIRWVDGLQASWLTLTLC

SEQ ID NO: 16 - DNA, 20 bp, Artificial Sequence
  oligonucleotide probe
atacgggaatggaactgttc

SEQ ID NO: 17 - DNA, 20 bp, Artificial Sequence
  oligonucleotide probe
tgtgggagtggtgcttcagt

SEQ ID NO: 18 - DNA, 20 bp, Artificial Sequence
  oligonucleotide probe
ttacgagcaggcccctgtac

SEQ ID NO: 19 - DNA, 20 bp, Artificial Sequence
  oligonucleotide probe
gtttgggaaggtagctcgcc

SEQ ID NO: 20 - DNA, 20 bp, Artificial Sequence
  oligonucleotide probe
gctaggcatgccctcctccc

SEQ ID NO: 21 - protein, 10 aa, Homo sapiens
LVYGNGTVLC

SEQ ID NO: 22 - protein, 10 aa, Homo sapiens
TSVGVVLQSP

SEQ ID NO: 23 - protein, 10 aa, Homo sapiens
EDYEQAPVLD

SEQ ID NO: 24 - protein, 10 aa, Homo sapiens
IVFGKVARRH

SEQ ID NO: 25 - protein, 10 aa, Homo sapiens
ALLGMPSSHL

SEQ ID NO: 26 - DNA, 28 bp, Artificial Sequence
  oligonucleotide primer
tacaaaatggaaaatggcagtgagtacc

SEQ ID NO: 27 - DNA, 23 bp, Artificial Sequence
  oligonucleotide primer
ggggtacactgggttggtcaaag

SEQ ID NO: 28 - DNA, 24 bp, Artificial Sequence
  oligonucleotide primer
atccgcttcgacatcattgtgttt

SEQ ID NO: 29 - DNA, 25 bp, Artificial Sequence
  oligonucleotide primer
ctctctgttctttgatggggctgtg

SEQ ID NO: 30 - DNA, 45 bp, Artificial Sequence
  oligonucleotide primer
atgaactgcatatccgacttcttcacctatgagaccaccaagtcg

SEQ ID NO: 31 - DNA, 48 bp, Artificial Sequence
  oligonucleotide primer
ctagtggcctatggagaaggcccccgaatcggtggactgcttctccgc

SEQ ID NO: 32 - DNA, 45 bp, Artificial Sequence
  oligonucleotide primer
atggcgggctgctgcgccgcgctggcggccttcctgttcgagtac

SEQ ID NO: 33 - DNA, 45 bp, Artificial Sequence
  oligonucleotide primer
tcactggtccagctcactagcaagaccctgctcgtaatcttccac

(19) United States
(12) Patent Application Publication
(10) Pub. No.: US 2006/0121508 A1
(43) Pub. Date: Jun. 8, 2006

(54) Alternatively spliced isoforms of purinergic receptor P2X, ligand-gated ion channel, subunits 3 and 4 (P2RX3, P2RX4)

(76) Inventors: Philip W. Garrett-Engele, Seattle, WA (US); Christopher K. Raymond, Seattle, WA (US)

Correspondence Address: R. Douglas Bradley;Merck & Co., Inc., P.O. Box 2000 - Patent Department RY60-30, Rahway, NJ, 07065-0907, US

(21) Appl. No.: 11/284,978

(22) Filed: Nov. 22, 2005

Related U.S. Application Data
(60) Provisional application No. 60/633,008, filed on Dec. 3, 2004.

(51) Int. Cl. C12Q 1/68; G01N 33/53; C07H 21/04; C12P 21/06; C07K 14/705; C07K 16/28

(52) U.S. Cl. 435/6; 435/7.1; 435/69.1; 435/320.1; 435/325; 530/350; 530/388.22; 536/23.5

(57) ABSTRACT

The present invention features nucleic acids and polypeptides encoding novel splice variant isoforms of purinergic receptor P2X, ligand-gated ion channel, subunits 3 and 4 (P2RX3, P2RX4). The polynucleotide sequences of P2RX3sv1 and P2RX3sv2 are provided by SEQ ID NO 6 and SEQ ID NO 8, respectively. The polynucleotide sequences of P2RX4sv1, P2RX4sv2, and P2RX4sv3 are provided by SEQ ID NO 10, SEQ ID NO 12, and SEQ ID NO 14, respectively. The amino acid sequences of P2RX3sv1 and P2RX3sv2 are provided by SEQ ID NO 7 and SEQ ID NO 9, respectively. The amino acid sequences of P2RX4sv1, P2RX4sv2, and P2RX4sv3 are provided by SEQ ID NO 11, SEQ ID NO 13, and SEQ ID NO 15, respectively. The present invention also provides methods for using P2RX3sv1, P2RX3sv2, P2RX4sv1, P2RX4sv2, and P2RX4sv3 polynucleotides and proteins to screen for compounds that bind to P2RX3sv1, P2RX3sv2, P2RX4sv1, P2RX4sv2, and P2RX4sv3, respectively.

This application claims priority to U.S. Provisional Patent Application Ser. No. 60/633,008 filed on Dec. 3, 2004, which is incorporated by reference herein in its entirety.

### BACKGROUND OF THE INVENTION

Adenosine triphosphate (ATP) has a fundamental role within cells as the universal energy source. In 1970s, Burnstock hypothesized that ATP could also act as an extracellular signal in nerve-mediated responses of gastrointestinal and bladder smooth muscles. Burnstock also proposed that ATP could act as a cotransmitter in both peripheral and central nerves (reviewed by Burnstock, 2001, Trends Pharmacol. Sci. 22:182-188). Initially, this concept met considerable resistance. However, today it is generally accepted that ATP exerts extracellular physiological roles through purinoceptors on target cells.

Cells release ATP as a result of a variety of conditions: tissue trauma, inflammation, visceral distension, hypoxia, acidosis, osmotic shock, mechanical stimulation, and tumor presence to name a few (reviewed in Bodin and Burnstock, 2001, Neurochem Res. 26:959-969). Extracellular ATP levels are controlled by ecto-nucleotideases. Thus, ATP is believed to be a local mediator, acting upon purinoceptors within the tissue environment before being modified by ecto-nucleotideases (reviewed in Schweibert and Zsembery, 2003, Biochim. Biophys. Acta 1615:7-32). Purinoceptors are divided into two subclasses: P1, which responds to adenosine, and P2, which responds to ATP. The P2 receptors are further distinguished into two subtypes. P2X receptors are cation-permeable ion channels gated by ATP and some other related nucleotides. P2Y receptors are coupled to heterotrimeric G proteins and phospholipases (reviewed in Schweibert and Zsembery, 2003, Biochim. Biophys. Acta 1615:7-32).

At least seven genes for P2X receptor subunits (P2RX1-P2RX7) have been identified to date. P2RX1 cDNA was cloned from the smooth muscle of the rat vas deferens, and P2RX2 cDNA was cloned from PC12 cells (Valera et al., 1994, Nature 371:516-519; Brake et al., 1994, Nature 371:519-523). The other five P2X receptors (P2RX3-P2RX7) were identified in rat neuronal cDNA libraries by searching for homologous sequences to P2RX1 and P2RX2 (Lewis et al., 1995, Nature 377:432-435; Chen et al., 1995, Nature 377:428-431; Buell et al. 1996, EMBO J. 15:55-62; Seguela et al., 1996, J. Neurosci. 16:448-455; Bo et al., 1995, FEBS Lett. 375:129-133; Soto et al., 1996, Proc. Natl. Acad. Sci. USA 93:3684-3688; Wang et al., 1996, Biochem. Biophys. Res. Commun. 220:196-202; Collo et al., 1996, J. Neurosci. 16:2495-2507; Garcia-Guzman et al., 1996, FEBS Lett. 388:123-127; Soto et al., 1996, Biochem. Biophys. Res. Commun. 223:456-460; Surprenant et al., 1996, Science 272:735-738). Garcia-Guzman et al. (1997, Mol. Brain Res. 47:59-66) cloned the human P2RX3 cDNA from human heart. The human P2RX4 cDNA was initially isolated from human brain (Garcia-Guzman et al., 1997, Mol. Pharmacol. 51:109-118). Human purinergic receptors P2X have also been disclosed (U.S. Pat. No. 6,214,581; U.S. Pat. No. 6,242,216).

All the P2X receptors have been found in the central and peripheral nervous systems, and studies on the localization of P2X receptors have found P2RX1-P2RX6 in nervous structures involved in nociceptive transmission (reviewed in Chizh and Liles, 2001, Pharmacol Rev. 53:553-568). In contrast to the distribution of the rat P2RX3 transcript, which was detected exclusively in the sensory neurons of trigeminal, dorsal root and nodose ganglia (Chen et al., 1995, Nature 377:428-431), the human P2RX3 transcript was detected in human spinal cord and heart. Expression analysis in both rat and human tissues has determined wide tissue distribution of P2RX4 transcripts in addition to the brain (Soto et al., 1996, Proc. Natl. Acad. Sci. USA 93:3684-3688; Garcia-Guzman et al., 1997, Mol. Pharmacol. 51:109-118).

The full length P2X receptor gene family has 11-13 exons and shares a common gene structure with well-conserved intron/exon boundaries. The P2X subunit proteins have two hydrophobic regions with the required length to traverse the plasma membrane (residues 30-50 and 330-353 with respective to the rat P2RX2 receptor). Between the hydrophobic transmembrane regions lies the bulk of the protein, which is primarily extracellular. Features of the extracellular loop include the conservation of 10 cysteine residues, which may contribute to the tertiary structure of the protein by disulfide bond formation, and multiple glycosylation sites. The short amino and carboxy termini are cytoplasmic (Newbolt et al., 1998, J. Biol. Chem. 273:15177-15182). The carboxy terminal region sequences vary considerably (reviewed by North, 2002, Physiol Rev. 82:1013-1067; Schwiebert and Zsembery, 2003, Biochim. Biophys. Acta 1615:7-32). P2RX3 gene has 12 exons and encodes a 397 amino acid protein. The P2RX4 gene has 12 exons and encodes a 388 amino acid protein.

P2X receptors are rapidly activated, nonselective cationic channels that are activated by ATP. P2RX4 is permeable to Na⁺, K⁺, and Ca²⁺ upon activation (Soto et al., 1996, Proc. Natl. Acad. Sci. USA 93:3684-3688; Garcia-Guzman et al., 1997, Mol Pharmacol. 51:109-118). P2RX3 exhibits considerably lower permeability for Ca²⁺ (Virginio et al., 1998, J. Physiol. 510:27-35). Individual P2X receptor subunits differ in their rates of desensitization in the continuous presence of ATP. P2RX3 receptors rapidly desensitize during prolonged exposure to ATP while P2RX4 receptors show slower desensitization (reviewed in Chizh and illes, 2001, Pharmacol. Rev. 53:553-568; North, 2002, Physiol. Rev. 81:1013-1067). Recovery of P2RX3 desensitization can be accelerated by higher Ca²⁺ concentrations (Cook et al., 1998, J. Neurosci. 18:9238-9244). The mechanism by which P2X receptors form the ionic pore or bind ATP is not known. Evidence suggests that P2X receptors may form trimeric complexes (Stoop et al. 1999, Mol. Pharmacol. 56:973-981; Nicke et al., 1998, EMBO J 17:3016-3028). Different cloned P2X receptor subunits may also combine to form functional heteromeric receptors, but the stoichiometry of the native P2X purinergic receptor remains unresolved at present (North, 2002, Physiol. Rev. 81:1013-1067).

Many splice variants of the P2X subunits have been described (reviewed in North, 2002, Physiol. Rev. 82:1013-1067). Koshimizu et al. (1998, Mol. Endocrinol. 12:901-913) found six different isoforms of P2RX2 in rat pituitary cells and characterized their function. Functional analysis of these splice variants determined that alterations in the extracellular loop resulted in nonfunctional channels while alteration of the C-terminus resulted in retention of channel activity. Reference transcript AF084975 refers to an alternatively spliced isoform of rat P2RX3 which is missing exon 3. Dhulipala et al. (1998, Gene 207:259-266) identified a human P2RX4 variant which possesses a 35 amino acid region with homology to HSP-90 in place of exons 1 and 2 and appears to be non-functional. Additional P2RX4 splice variants with alterations in the extracellular region were identified in humans (Carpenter et al., 1999, Neurosci. Lett. 273:183-186; PCT Publication WO97/41222). One of these variants, which has a deletion of 130 amino acids including 6 of the 10 conserved cysteine residues, does not form a functional channel. A P2RX4 splice variant has also been identified in mouse which lacks exon 6, affecting the extracellular domain. This mouse P2RX4 splice variant forms poorly functional homomeric receptors. Functional studies have demonstrated that P2X receptor subunit splice variants may interact with other subunits and alter the functions of heteromeric receptors (Carpenter et al., 1999, Neurosci. Lett. 273:183-186; Townsend-Nicholson et al., 1999, Mol Brain Res. 64:246-254; Koshimizu et al., 1998, Mol. Endocrinol. 12:901-913).

The distribution of purinergic receptors P2X in pain relevant neuronal structures suggest their involvement in pain mechanisms (reviewed in Chizh and Illes, 2001, Pharmacol. Rev. 53:553-568). Nerve fibers in the suburothelial plexus in mouse bladder showed abundant immuno-staining for P2RX3 (Vlaskovska et al., 2001, J. Neurosci. 21:5670-5677). Although ATP was released similarly in response to bladder extension in both normal and P2RX3 knockout mice, the bladder afferent nerve activity showed attenuated response to distension in the P2RX3 knockout mice. Zhong et al. (2001, Eur. J. Neurosci. 14:1784-1792) examined the changes in P2X receptor responses on DRG and nodose neurons in P2RX3 knockout mice. DRG neurons in P2RX3^{-/-} mice lacked rapidly desensitizing response to ATP, characteristic of P2RX3 receptors, and both DRG and nodose neurons from the knockout mice failed to respond to agonist αβmeATP. P2RX3^{-/-} mice also showed reduced formalin-induced pain behavior, and a deficit in the ability to code the intensity of non-noxious ‘warming’ stimuli (Souslova et al., 2000, Nature 407:1015-1017). Antisense oligonucleotides and siRNA specific for P2RX3 decreased chronic neuropathic pain in rats (Honore et al., 2002, Pain 99:11-19; Dorn et al., 2004, Nucleic Acids Res. 32:e49). P2RX3 function is also implicated in transmission of visceral pain (Honore et al., 2002, Pain 96:99-105). It has been shown that P2RX4 is involved in tactile allodynia, long-lasting pain hypersensitivity to innocuous stimuli, after nerve injury (Tsuda et al., 2003, Nature 424:778-783; Inoue et al., 2004, J. Pharmacol. 94:112-114). Following peripheral nerve injury, rats displayed marked tactile allodynia and increased level of P2RX4 protein in the ipsilateral spinal cord, in microglia particularly. Inhibition of P2RX4 by a pharmacological agent or by an antisense oligodeoxynucleotide reversed or suppressed tactile allodynia after nerve injury Additionally, intrathecal administration of P2RX4-stimulated microglia into naïve rats was sufficient to produce tactile allodynia (Tsuda et al., 2003, Nature 424:778-783).

The number of pharmacological compounds to study P2X receptor function is limited. (reviewed by Chizh and Illes, 2001, Pharmacol Rev. 53:553-568; North, 2002, Physiol. Rev. 82:1013-1067). 2-Methylthio-ATP (2-MeSATP) and α,β-methylene-ATP (α,β-meATP) are potent agonists for P2RX3 (Garcia-Guzman et al., 1997, Mol Brain Res. 47:59-66). Diadensoine pentaphosphate (Ap5A) is also a full agonist for P2RX3 and partial agonist for P2RX1 (Wildman et al., 1999, Eur. J. Pharmacol. 367:119-123). P2RX3 is blocked by suramin at high concentrations, a general P2X receptor antagonist with the exception of P2RX6 and rat P2RX4 (reviewed by Burnstock, 2001, Trends Pharmacol. Sci. 22:182-188). Pyridoxalphosphate-6-azophenyl-2′,4′-disulfonic acid (PPADS) and (TNP-ATP) are also weak, nonselective antagonists for P2RX1 and P2RX3 receptors (reviewed by Chizh and Illes, 2001, Pharmacol Rev. 53:553-568; North, 2002, Physiol. Rev. 82:1013-1067). The human and rat P2RX4 receptors differ in their sensitivity to the general P2X receptor antagonists, suramin and PPADS. Recent work by Xiong et al. (2004, Neurosci. Lett. 367:197-200) suggests that an extracellular histidine residue is involved in regulating the antagonist sensitivity of P2RX4 receptors. Trace metals, such as copper and zinc, differentially modulate purinergic P2X receptors (Xiong et al., 1999, J. Neurophysiol. 81:2088-2094; Wildman et al., 1999, Br. J. Pharmacol. 128:486-492). Recently, A-317491 (Jarvis et al., 2002, Proc. Natl. Acad. Sci. USA 99:17179-17184) was shown to be a potent and selective non-nucleotide antagonist of P2RX3 and was effective in reducing chronic inflammatory and neuropathic pain in rats.

Unfortunately, the study of P2X receptors is hindered by the lack of receptor subtype-specific agonists and antagonists (reviewed in Chizh and Illes, 2001, Pharacol. Rev. 53:553-568; North, 2002, Physiol. Rev. 82:1013-1067). Because of the multiple therapeutic values of drugs targeting purinergic receptors P2X, including P2RX3 and P2RX4, there is a need in the art for compounds that selectively bind to isoforms of P2RX3 and P2RX4. The present invention is directed toward novel P2RX3 isoforms (P2RX3sv1, P2RX3sv2) and P2RX4 (P2RX4sv1, P2RX4sv2, and P2RX4sv3) and uses thereof.

### BRIEF DESCRIPTION OF THE DRAWINGS

FIG. 1A illustrates the exon structure of human P2RX3 mRNA corresponding to the known reference form of P2RX3 mRNA (labeled NM_{—}002559) and the exon structure corresponding to the inventive splice variant transcripts (labeled P2RX3sv1 and P2RX3sv2). FIG. 1B depicts the nucleotide sequences of the exon junctions resulting from the splicing of exon 9 to exon 11 [SEQ ID NO 1], in the case of P2RX3sv1 mRNA, and the splicing of exon 10 to novel intron 10′ [SEQ ID NO 2] in the case of P2RX3sv2 mRNA. In FIG. 1B, in the case of the P2RX3sv1 exon 9-exon 11 splice junction sequence [SEQ ID NO 1], the nucleotides shown in italics represent the 20 nucleotides at the 3′ end of exon 9 and the nucleotides shown in underline represent the 20 nucleotides at the 5′ end of exon 11; and in the case of P2RX3sv2 exon 10-intron 10′ splice junction sequence [SEQ ID NO 2], the nucleotides shown in italics represent the 20 nucleotides at the 3′ end of exon 10 and the nucleotides shown in underline represent the 20 nucleotides at the 5′ end of intron 10′.

FIG. 2A illustrates the exon structure of human P2RX4 mRNA corresponding to the known reference form of P2RX4 mRNA (labeled NM_{—}002560) and the exon structure corresponding to the inventive splice variant transcripts (labeled P2RX4sv1, P2RX4sv2, and P2RX4sv3). FIG. 2B depicts the nucleotide sequences of the exon junctions resulting from the splicing of exon 11 to intron 11′ [SEQ ID NO 3] in the case of P2RX4sv1 mRNA, the splicing of exon 9 to intron 9 [SEQ ID NO 4] in the case of P2RX4sv2, and the splicing of exon 10 to exon 11 with 5′ extension [SEQ ID NO 5] in the case of P2RX4sv3. In FIG. 2B, in the case of P2RX4sv1 exon 11-intron 11′ splice junction sequence [SEQ ID NO 3], the nucleotides shown in italics represent the 20 nucleotides at the 3′ end of exon 11 and the nucleotides shown in underline represent the 20 nucleotides at the 5′ end of intron 11′; in the case of P2RX4sv2 exon 9-intron 9 splice junction sequence [SEQ ID NO 4], the nucleotides shown in italics represent the 20 nucleotides at the 3′ end of exon 9 and the nucleotides shown in underline represent the 20 nucleotides at the 5′ end of intron 9; and in the case of P2RX4sv3 exon 10-exon 11 5′ extension splice junction sequence [SEQ ID NO 5], the nucleotides shown in italics represent the 20 nucleotides at the 3′ end of exon 10 and the nucleotides shown in underline represent the 20 nucleotides at the 5′ end of exon 11 5′ extension.

### SUMMARY OF THE INVENTION

RT-PCR experiments have been used to identify and confirm the presence of novel splice variants of human P2RX3 and P2RX4 mRNA. More specifically, the present invention features polynucleotides encoding different protein isoforms of P2RX3 and P2RX4. A polynucleotide sequence encoding P2RX3sv1 is provided by SEQ ID NO 6. An amino acid sequence for P2RX3sv1 is provided by SEQ ID NO 7. A polynucleotide sequence encoding P2RX3sv2 is provided by SEQ ID NO 8. An amino acid sequence for P2RX3sv2 is provided by SEQ ID NO 9. A polynucleotide sequence encoding P2RX4sv1 is provided by SEQ ID NO 10. An amino acid sequence for P2RX4sv1 is provided by SEQ ID NO 11. A polynucleotide sequence encoding P2RX4sv2 is provided by SEQ ID NO 12. An amino acid sequence for P2RX4sv2 is provided by SEQ ID NO 13. A polynucleotide sequence encoding P2RX4sv3 is provided by SEQ ID NO 14. An amino acid sequence for P2RX4sv3 is provided by SEQ ID NO 15.

Thus, a first aspect of the present invention describes a purified P2RX3sv1 encoding nucleic acid, a purified P2RX3sv2 encoding nucleic acid, a purified P2RX4sv1 encoding nucleic acid, a P2RX4sv2 encoding nucleic acid, a P2RX4sv3 encoding nucleic acid, and their respective complementary sequences. The P2RX3sv1 encoding nucleic acid comprises SEQ ID NO 6. The P2RX3sv2 encoding nucleic acid comprises SEQ ID NO 8. The P2RX4sv1 encoding nucleic acid comprises SEQ ID NO 10. The P2RX4sv2 encoding nucleic acid comprises SEQ ID NO 12. The P2RX4sv3 encoding nucleic acid comprises SEQ ID NO 14. Reference to the presence of one region does not indicate that another region is not present. For example, in different embodiments the inventive nucleic acid can comprise, consist, or consist essentially of an encoding nucleic acid sequence of SEQ ID NO 6; can comprise, consist, or consist essentially of the nucleic acid sequence of SEQ ID NO 8; can comprise, consist, or consist essentially of the nucleic acid sequence of SEQ ID NO 10; can comprise, consist, or consist essentially of the nucleic acid sequence of SEQ ID NO 12; or alternatively can comprise, consist, or consist essentially of the nucleic acid sequence of SEQ ID NO 14.

Another aspect of the present invention describes a purified P2RX3sv1 polypeptide that can comprise, consist or consist essentially of the amino acid sequence of SEQ ID NO 7. An additional aspect describes a purified P2RX3sv2 polypeptide that can comprise, consist, or consist essentially of the amino acid sequence of SEQ ID NO 9. An additional aspect describes a purified P2RX4sv1 polypeptide that can comprise, consist, or consist essentially of the amino acid sequence of SEQ ID NO 11. An additional aspect describes a purified P2RX4sv2 polypeptide that can comprise, consist, or consist essentially of the amino acid sequence of SEQ ID NO 13. An additional aspect describes a purified P2RX4sv3 polypeptide that can comprise, consist, or consist essentially of the amino acid sequence of SEQ ID NO 15.

Another aspect of the present invention describes expression vectors. In one embodiment of the invention, the inventive expression vector comprises a nucleotide sequence encoding a polypeptide comprising, consisting, or consisting essentially of SEQ ID NO 7, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 9, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 11, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 13, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 15, wherein the nucleotide sequence is transcriptionally coupled to an exogenous promoter.

Alternatively, the nucleotide sequence comprises, consists, or consists essentially of SEQ ID NO 6, and is transcriptionally coupled to an exogenous promoter. In another embodiment, the nucleotide sequence comprises, consists, or consists essentially of SEQ ID NO 8, and is transcriptionally coupled to an exogenous promoter. In another embodiment, the nucleotide sequence comprises, consists, or consists essentially of SEQ ID NO 10, and is transcriptionally coupled to an exogenous promoter. In another embodiment, the nucleotide sequence comprises, consists, or consists essentially of SEQ ID NO 12, and is transcriptionally coupled to an exogenous promoter. In another embodiment, the nucleotide sequence comprises, consists, or consists essentially of SEQ ID NO 14, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 6, SEQ ID NO 8, SEQ ID NO 10, SEQ ID NO 12, or SEQ ID NO 14, or a nucleotide sequence encoding a polypeptide comprising, consisting, or consisting essentially of an amino acid sequence of SEQ ID NO 7, SEQ ID NO 9, SEQ ID NO 11, SEQ ID NO 13, or SEQ ID NO 15, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing P2RX3sv1, P2RX3sv2, P2RX4sv1, P2RX4sv2, or P2RX4sv3 polypeptide comprising SEQ ID NO 7, SEQ ID NO 9, SEQ ID NO 11, SEQ ID NO 13, or SEQ ID NO 15, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to P2RX3sv1 as compared to one or more purinergic receptor P2X polypeptides that are not P2RX3sv1. In another embodiment, a purified antibody preparation is provided comprising an antibody that binds selectively to P2RX3sv2 as compared to one or more purinergic receptor P2X polypeptides that are not P2RX3sv2. In another embodiment, a purified antibody preparation is provided comprising an antibody that binds selectively to P2RX4sv1 as compared to one or more purinergic receptor P2X polypeptides that are not P2RX4sv1. In another embodiment, a purified antibody preparation is provided comprising an antibody that binds selectively to P2RX4sv2 as compared to one or more purinergic receptor P2X polypeptides that are not P2RX4sv2. In another embodiment, a purified antibody preparation is provided comprising an antibody that binds selectively to P2RX4sv3 as compared to one or more purinergic receptor P2X polypeptides that are not P2RX4sv3.

Another aspect of the present invention provides a method of screening for a compound that binds to P2RX3sv1, P2RX3sv2, or fragments thereof. In one embodiment, the method comprises the steps of: (a) expressing a polypeptide comprising the amino acid sequence of SEQ ID NO 7 or a fragment thereof from recombinant nucleic acid; (b) providing to said polypeptide a labeled P2RX3 ligand that binds to said polypeptide and a test preparation comprising one or more test compounds; (c) and measuring the effect of said test preparation on binding of said test preparation to said polypeptide comprising SEQ ID NO 7. Alternatively, this method could be performed using SEQ ID NO 9 instead of SEQ ID NO 7.

Another aspect of the present invention provides a method of screening for a compound that binds to P2RX4sv1, P2RX4sv2, P2RX4sv3, or fragments thereof. In one embodiment, the method comprises the steps of: (a) expressing a polypeptide comprising the amino acid sequence of SEQ ID NO II or a fragment thereof from recombinant nucleic acid; (b) providing to said polypeptide a labeled P2RX4 ligand that binds to said polypeptide and a test preparation comprising one or more test compounds; (c) and measuring the effect of said test preparation on binding of said test preparation to said polypeptide comprising SEQ ID NO 11. Alternatively, this method could be performed using SEQ ID NO 13 or SEQ ID NO 15, instead of SEQ ID NO 11.

In another embodiment of the method, a compound is identified that binds selectively to P2RX3sv1 polypeptide as compared to one or more purinergic receptor P2X polypeptides that are not P2RX3sv1. This method comprises the steps of: providing a P2RX3sv1 polypeptide comprising SEQ ID NO 7; providing a purinergic receptor P2X polypeptide that is not P2RX3sv1; contacting said P2RX3sv1 polypeptide and said purinergic receptor P2X polypeptide that is not P2RX3sv1 with a test preparation comprising one or more test compounds; and determining the binding of said test preparation to said P2RX3sv1 polypeptide and to purinergic receptor P2X polypeptide that is not P2RX3sv1, wherein a test preparation that binds to said P2RX3sv1 polypeptide but does not bind to said purinergic receptor P2X polypeptide that is not P2RX3sv1 contains a compound that selectively binds said P2RX3sv1 polypeptide. Alternatively, the same method can be performed using P2RX3sv2 polypeptide comprising, consisting, or consisting essentially of SEQ ID NO 9. Alternatively, the same method can be performed using P2RX4sv1 polypeptide comprising, consisting, or consisting essentially of SEQ ID NO 11. Alternatively, the same method can be performed using P2RX4sv2 polypeptide comprising, consisting, or consisting essentially of SEQ ID NO 13. Alternatively, the same method can be performed using P2RX4sv3 polypeptide comprising, consisting, or consisting essentially of SEQ ID NO 15.

In another embodiment of the invention, a method is provided for screening for a compound able to bind to or interact with a P2RX3sv1 protein or a fragment thereof comprising the steps of: expressing a P2RX3sv1 polypeptide comprising SEQ ID NO 7 or a fragment thereof from a recombinant nucleic acid; providing to said polypeptide a labeled P2RX3 ligand that binds to said polypeptide and a test preparation comprising one or more compounds; and measuring the effect of said test preparation on binding of said labeled P2RX3 ligand to said polypeptide, wherein a test preparation that alters the binding of said labeled P2RX3 ligand to said polypeptide contains a compound that binds to or interacts with said polypeptide. In an alternative embodiment, the method is performed using P2RX3sv2 polypeptide comprising, consisting, or consisting essentially of SEQ ID NO 9 or a fragment thereof.

In another embodiment of the invention, a method is provided for screening for a compound able to bind to or interact with a P2RX4sv1 protein or a fragment thereof comprising the steps of: expressing a P2RX4sv1 polypeptide comprising SEQ ID NO 11 or a fragment thereof from a recombinant nucleic acid; providing to said polypeptide a labeled P2RX4 ligand that binds to said polypeptide and a test preparation comprising one or more compounds; and measuring the effect of said test preparation on binding of said labeled P2RX4 ligand to said polypeptide, wherein a test preparation that alters the binding of said labeled P2RX4 ligand to said polypeptide contains a compound that binds to or interacts with said polypeptide. In an alternative embodiment, the method is performed using P2RX4sv2 polypeptide comprising, consisting, or consisting essentially of SEQ ID NO 13 or a fragment thereof. In an alternative embodiment, the method is performed using P2RX4sv3 polypeptide comprising, consisting, or consisting essentially of SEQ ID NO 15 or a fragment thereof.

Another aspect of the present invention provides a method of screening for a compound that binds to one or more purinergic receptor P2X polypeptides that are not P2RX3sv1. This method comprises the steps of: providing a P2RX3sv1 polypeptide comprising SEQ ID NO 7; providing a purinergic receptor P2X polypeptide that is not P2RX3sv1; contacting said P2RX3sv1 polypeptide and purinergic receptor P2X polypeptide that is not P2RX3sv1 with a test preparation comprising one or more test compounds; and determining the binding of said test preparation to said P2RX3sv1 polypeptide and to said purinergic receptor P2X polypeptide that is not P2RX3sv1, wherein a test preparation that binds to said purinergic receptor P2X polypeptide that is not P2RX3sv1 but not to said P2RX3sv1 polypeptide contains a compound that selectively binds said purinergic receptor P2X polypeptide. Alternatively, the same method can be performed using P2RX3sv2 polypeptide comprising, consisting, or consisting essentially of SEQ ID NO 9. Alternatively, the same method can be performed using P2RX4sv1 polypeptide comprising, consisting, or consisting essentially of SEQ ID NO 11. Alternatively, the same method can be performed using P2RX4sv2 polypeptide comprising, consisting, or consisting essentially of SEQ ID NO 13. Alternatively, the same method can be performed using P2RX4sv3 polypeptide comprising, consisting, or consisting essentially of SEQ ID NO 15.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “P2RX3” refers to human purinergic receptor P2X, ligand-gated ion channel, subunit 3 protein (P2RX3), also known as P2X3, (NP_{—}002550). In contrast, reference to a P2RX3 isoform, includes NP_{—}002550 and other polypeptide isoform variants of P2RX3.

As used herein, “P2RX4” refers to human purinergic receptor P2X, ligand-gated ion channel, subunit 4 protein (P2RX4), also known as P2X4, (NP_{—}002551). In contrast, reference to a P2RX4 isoform, includes NP_{—}002551 and other polypeptide isoform variants of P2RX4.

As used herein, “P2RX3sv1” and “P2RX3sv2” refer to splice variant isoforms of human P2RX3 protein, wherein the splice variants have the amino acid sequence set forth in SEQ ID NO 7 (for P2RX3sv1) and SEQ ID NO 9 (for P2RX3sv2).

As used herein, “P2RX4sv1”, “P2RX4sv2”, and “P2RX4sv3” refer to splice variant isoforms of human P2RX4 protein, wherein the splice variants have the amino acid sequence set forth in SEQ ID NO 11 (for P2RX4sv1), SEQ ID NO 13 (for P2RX4sv2), and SEQ ID NO 15 (for P2RX4sv3).

As used herein, “P2RX3” refers to polynucleotides encoding P2RX3.

As used herein, “P2RX4” refers to polynucleotides encoding P2RX4.

As used herein, “P2RX3sv1” refers to polynucleotides encoding P2RX3sv1 having an amino acid sequence set forth in SEQ ID NO 7.

As used herein, “P2RX3sv2” refers to polynucleotides encoding P2RX3sv2 having an amino acid sequence set forth in SEQ ID NO 9.

As used herein, “P2RX4sv1” refers to polynucleotides encoding P2RX4sv1 having an amino acid sequence set forth in SEQ ID NO 11.

As used herein, “P2RX4sv2” refers to polynucleotides encoding P2RX4sv2 having an amino acid sequence set forth in SEQ ID NO 13.

As used herein, “P2RX4sv3” refers to polynucleotides refers to polynucleotides encoding P2RX4sv3 having an amino acid sequence set forth in SEQ ID NO 15.

As used herein, a “purinergic receptor P2X” is any isoform of any purinergic receptor P2X, ligand-gated ion channel from any organism, including but not limited to human P2RX1 (P2X1), P2RX2 (P2X2), P2RX3 (P2X3), P2RX4 (P2X4), P2RX5 (P2X5), P2RX6 (P2X6), and P2RX7 (P2X7).

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human P2RX3sv1 and P2RX3sv2 that are alternatively spliced isoforms of P2RX3, and to the amino acid sequences encoding these proteins. SEQ ID NO 6 and SEQ ID NO 8 are polynucleotide sequences representing exemplary open reading frames that encode the P2RX3sv1 and P2RX3sv2 proteins, respectively. SEQ ID NO 7 shows the polypeptide sequence of P2RX3sv1. SEQ ID NO 9 shows the polypeptide sequence of P2RX3sv2.

The present invention also relates to the nucleic acid sequences encoding human P2RX4sv1, P2RX4sv2, and P2RX4sv3 that are alternatively spliced isoforms of P2RX4, and to the amino acid sequences encoding these proteins. SEQ ID NO 10, SEQ ID NO 12, and SEQ ID NO 14 are polynucleotide sequences representing exemplary open reading frames that encode the P2RX4sv1, P2RX4sv2, and P2RX4sv3 proteins, respectively. SEQ ID NO 11 shows the polypeptide sequence of P2RX4sv1. SEQ ID NO 13 shows the polypeptide sequence of P2RX4sv2. SEQ ID NO 15 shows the polypeptide sequence of P2RX4sv3.

P2RX3sv1 and P2RX3sv2 polynucleotide sequences encoding P2RX3sv1 and P2RX3sv2 proteins, as exemplified and enabled herein include a number of specific, substantial and credible utilities. For example, P2RX3sv1 and P2RX3sv2 encoding nucleic acids were identified in an mRNA sample obtained from a human source (see Example 1). Such nucleic acids can be used as hybridization probes to distinguish between cells that produce P2RX3sv1 and P2RX3sv2 transcripts from human or non-human cells (including bacteria) that do not produce such transcripts. Similarly, antibodies specific for P2RX3sv1 or P2RX3sv2 can be used to distinguish between cells that express P2RX3sv1 or P2RX3sv2 from human or non-human cells (including bacteria) that do not express P2RX3sv1 or P2RX3sv2.

P2RX4sv1, P2RX4sv2, and P2RX4sv3 polynucleotide sequences encoding P2RX4sv1, P2RX4sv2, and P2RX4sv3 proteins, as exemplified and enabled herein include a number of specific, substantial and credible utilities. For example, P2RX4sv1, P2RX4sv2, and P2RX4sv3 encoding nucleic acids were identified in an mRNA sample obtained from a human source (see Example 1). Such nucleic acids can be used as hybridization probes to distinguish between cells that produce P2RX4sv1, P2RX4sv2, and P2RX4sv3 transcripts from human or non-human cells (including bacteria) that do not produce such transcripts. Similarly, antibodies specific for P2RX4sv1, P2RX4sv2, or P2RX4sv3 can be used to distinguish between cells that express P2RX4sv1, P2RX4sv2, or P2RX4sv3 from human or non-human cells (including bacteria) that do not express P2RX4sv1, P2RX4sv2, or P2RX4sv3.

The importance of P2RX3 and P2RX4 as a drug target for a range of neurological disorders including chronic inflammatory pain, acute pain, neuropathic pain, visceral pain, and tactile allodynia, is evidenced by the reduction of these neurological phenotypes in rodents with purinergic receptor P2X deficiencies or receiving P2X inhibitors (Vlaskovska et al., 2001, J. Neurosci. 21:5670-5677; Zhong et al., 2001, Eur. J. Neurosci. 14:1784-1792; Souslova et al., 2000, Nature 407:1015-1017 Honore et al., 2002, Pain 99:11-19; Dorn et al., 2004, Nucleic Acids Res. 32:e49; Tsuda et al., 2003, Nature 424:778-783). Given the potential importance of P2RX3 and P2RX4 activity to the therapeutic management of a wide array of diseases, it is of value to identify P2RX3 and P2RX4 isoforms and identify P2RX3 and P2RX4-ligand compounds that are isoform specific, as well as compounds that are effective ligands for two or more different P2RX3 or P2RX4 isoforms, respectively, or purinergic receptor P2X isoforms. In particular, it may be important to identify compounds that are effective inhibitors of a specific P2RX3 isoform activity, yet do not bind to or interact with a plurality of different P2RX3 isoforms or purinergic receptor P2X isoforms. Compounds that bind to or interact with multiple P2RX3 isoforms may require higher drug doses to saturate multiple P2RX3-isoform binding sites and thereby result in a greater likelihood of secondary non-therapeutic side effects. Furthermore, biological effects could also be caused by the interaction of a drug with the P2RX3sv1 or P2RX3sv2 isoforms specifically. For the foregoing reasons, P2RX3sv1 and P2RX3sv2 proteins represent useful compound binding targets and have utility in the identification of new P2RX3-ligands and purinergic receptor P2X isoform-ligands exhibiting a preferred specificity profile and having greater efficacy for their intended use. Additionally, it may be important to identify compounds that are effective inhibitors of a specific P2RX4 isoform activity, yet do not bind to or interact with a plurality of different P2RX4 isoforms or purinergic receptor P2X isoforms. Compounds that bind to or interact with multiple P2RX4 isoforms may require higher drug doses to saturate multiple P2RX4-isoform binding sites and thereby result in a greater likelihood of secondary non-therapeutic side effects. Furthermore, biological effects could also be caused by the interaction of a drug with the P2RX4sv1, P2RX4sv2, or P2RX4sv3 isoforms specifically. For the foregoing reasons, P2RX4sv1, P2RX4sv2, and P2RX4sv3 proteins represent useful compound binding targets and have utility in the identification of new P2RX4-ligands and purinergic receptor P2X isoform-ligands exhibiting a preferred specificity profile and having greater efficacy for their intended use.

In some embodiments, P2RX3sv1, P2RX3sv2, P2RX4sv1, P2RX4sv2, and P2RX4sv3 activity is modulated by a ligand compound to achieve one or more of the following: prevent or reduce the risk of occurrence, or recurrence of neurological disorders including chronic inflammatory pain, acute pain, neuropathic pain, visceral pain, and tactile allodynia.

Compounds modulating P2RX3sv1, P2RX3sv2, P2RX4sv1, P2RX4sv2, or P2RX4sv3 include agonists, antagonists, and allosteric modulators. While not wishing to be limited to any particular theory of therapeutic efficacy, generally, but not always, P2RX3sv1 or P2RX3sv2 compounds will be used to modulate the activity of the P2RX3 purinergic receptor P2X, ligand-gated ion channel, subunit 3. While not wishing to be limited to any particular theory of therapeutic efficacy, generally, but not always, P2RX4sv1, P2RX4sv2, or P2RX4sv3 compounds will be used to modulate the activity of the P2RX4 purinergic receptor P2X, ligand-gated ion channel, subunit 4. The expression level of P2RX3 and P2RX4 have been shown to correlate with symptoms of interstitial cystitis, tactile allodynia, and other pain mechanisms (Sun and Chai, 2004, J. Urol. 171:448-452; Tsuda et al., 2003, Nature 424:778-783; Inoue et al., 2004, J. Pharmacol. Sci. 94:112-114; reviewed in Chizh and Illes, 2001, Pharmacol. Rev. 53:553-568). Therefore, agents that modulate P2RX3 activity may be used to achieve a therapeutic benefit for any disease or condition due to, or exacerbated by, P2RX3 ion channel activity. Additionally, agents that modulate P2RX4 activity may be used to achieve a therapeutic benefit for any disease or condition due to, or exacerbated by, P2RX4 ion channel activity.

P2RX3sv1, P2RX3sv2, P2RX4sv1, P2RX4sv2, or P2RX4sv3 activity can also be affected by modulating the cellular abundance of transcripts encoding P2RX3sv1, P2RX3sv2, P2RX4sv1, P2RX4sv2, or P2RX4sv3, respectively. Compounds modulating the abundance of transcripts encoding P2RX3sv1, P2RX3sv2, P2RX4sv1, P2RX4sv2, or P2RX4sv3 include a cloned polynucleotide encoding P2RX3sv1, P2RX3sv2, P2RX4sv1, P2RX4sv2, or P2RX4sv3, respectively, that can express P2RX3sv1, P2RX3sv2, P2RX4sv1, P2RX4sv2, or P2RX4sv3 in vivo, antisense nucleic acids targeted to P2RX3sv1, P2RX3sv2, P2RX4sv1, P2RX4sv2, or P2RX4sv3 transcripts, enzymatic nucleic acids, such as ribozymes, and RNAi nucleic acids, such as shRNAs or siRNAs, targeted to P2RX3sv1, P2RX3sv2, P2RX4sv1, P2RX4sv2, or P2RX4sv3 transcripts.

In some embodiments, P2RX3sv1 or P2RX3sv2 activity is modulated to achieve a therapeutic effect upon diseases in which regulation of P2RX3 is desirable. In some embodiments, P2RX4sv1, P2RX4sv2, or P2RX4sv3 activity is modulated to achieve a therapeutic effect upon diseases in which regulation of P2RX4 is desirable. For example, neurological disorders such as chronic inflammatory pain, acute pain, neuropathic pain, visceral pain, and tactile allodynia may be treated by modulating P2RX3sv1, P2RX3sv2, P2RX4sv1, P2RX4sv2, or P2RX4sv3 ion channel activity.

P2RX3sv1, P2RX3sv2, P2RX4sv1, P2RX4sv2, and P2RX4sv3 Nucleic Acids

P2RX3sv1 nucleic acids contain regions that encode for polypeptides comprising, consisting, or consisting essentially of SEQ ID NO 7. P2RX3sv2 nucleic acids contain regions that encode for polypeptides comprising, consisting, or consisting essentially of SEQ ID NO 9. P2RX4sv1 nucleic acids contain regions that encode for polypeptides comprising, consisting, or consisting essentially of SEQ ID NO 11. P2RX4sv2 nucleic acids contain regions that encode for polypeptides comprising, consisting, or consisting essentially of SEQ ID NO 13. P2RX4sv3 nucleic acids contain regions that encode for polypeptides comprising, consisting, or consisting essentially of SEQ ID NO 15. The P2RX3sv1, P2RX3sv2, P2RX4sv1, P2RX4sv2, and P2RX4sv3 nucleic acids have a variety of uses, such as use as a hybridization probe or PCR primer to identify the presence of P2RX3sv1, P2RX3sv2, P2RX4sv1, P2RX4sv2, or P2RX4sv3 nucleic acids, respectively; use as a hybridization probe or PCR primer to identify nucleic acids encoding for proteins related to P2RX3sv1, P2RX3sv2, P2RX4sv1, P2RX4sv2, or P2RX4sv3, respectively; and/or use for recombinant expression of P2RX3sv1, P2RX3sv2, P2RX4sv1, P2RX4sv2, or P2RX4sv3 polypeptides, respectively. In particular, P2RX3sv1 polynucleotides do not have the polynucleotide regions that consist of exon 10 of the P2RX3 gene. P2RX3sv2 polynucleotides retain an additional polynucleotide region that consists of intron 10′ of the P2RX3 gene. Intron 10′ consists of a portion of intron 10 of the P2RX3 gene. P2RX4sv1 polynucleotides have an additional polynucleotide region that consists of intron 11′. Intron 11′ consists of a portion of intron 11 of the P2RX4 gene. P2RX4sv2 polynucleotides have an additional polynucleotide region that consists of intron 9 of the P2RX4 gene. P2RX4sv3 polynucleotides have an additional polynucleotide region at the 5′ end of exon 11 that consists of a portion of intron 10 (exon 11 5′ extension) of the P2RX4 gene.

Regions in P2RX3sv1, P2RX3sv2, P2RX4sv1, P2RX4sv2, or P2RX4sv3 nucleic acid that do not encode for P2RX3sv1, P2RX3sv2, P2RX4sv1, P2RX4sv2, or P2RX4sv3, or are not found in SEQ ID NO 6, SEQ ID NO 8, SEQ ID NO 10, SEQ ID NO 12, or SEQ ID NO 14, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding P2RX3sv1, P2RX3sv2, P2RX4sv1, P2RX4sv2, or P2RX4sv3 related proteins from different sources. Obtaining nucleic acids encoding P2RX3sv1, P2RX3sv2, P2RX4sv1, P2RX4sv2, or P2RX4sv3 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

P2RX3sv1, P2RX3sv2, P2RX4sv1, P2RX4sv2, or P2RX4sv3 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

P2RX3sv1, P2RX3sv2, P2RX4sv1, P2RX4sv2, and P2RX4sv3 Probes

Probes for P2RX3sv1 or P2RX3sv2 contain a region that can specifically hybridize to P2RX3sv1 or P2RX3sv2 target nucleic acids, respectively, under appropriate hybridization conditions and can distinguish P2RX3sv1 or P2RX3sv2 nucleic acids from each other and from non-target nucleic acids, in particular P2RX3 polynucleotides containing exon 10 and lacking intron 10′. Probes for P2RX3sv1 or P2RX3sv2 can also contain nucleic acid regions that are not complementary to P2RX3sv1 or P2RX3sv2 nucleic acids.

Probes for P2RX4sv1, P2RX4sv2, or P2RX4sv3 contain a region that can specifically hybridize to P2RX4sv1, P2RX4sv2, or P2RX4sv3 target nucleic acids, respectively, under appropriate hybridization conditions and can distinguish P2RX4sv1, P2RX4sv2, or P2RX4sv3 nucleic acids from each other and from non-target nucleic acids, in particular P2RX4 polynucleotides lacking intron 11′, intron 9, and 5′ extension on exon 11. Probes for P2RX4sv1, P2RX4sv2, or P2RX4sv3 can also contain nucleic acid regions that are not complementary to P2RX4sv1, P2RX4sv2, or P2RX4sv3 nucleic acids.

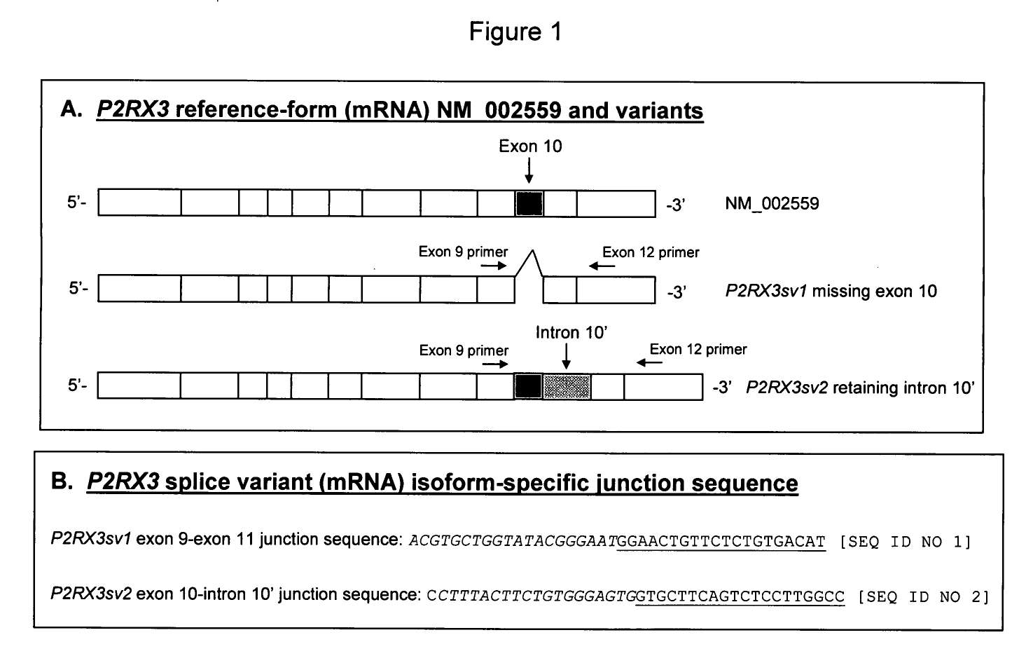

In embodiments where, for example, P2RX3sv1, P2RX3sv2, P2RX4sv1, P2RX4sv2, or P2RX4sv3 polynucleotide probes are used in hybridization assays to specifically detect the presence of P2RX3sv1, P2RX3sv2, P2RX4sv1, P2RX4sv2, or P2RX4sv3 polynucleotides in samples, the P2RX3sv1, P2RX3sv2, P2RX4sv1, P2RX4sv2, or P2RX4sv3 polynucleotides comprise at least 20 nucleotides of the P2RX3sv1, P2RX3sv2, P2RX4sv1, P2RX4sv2, or P2RX4sv3 sequence that correspond to the respective novel exon junction or novel polynucleotide regions. In particular, for detection of P2RX3sv1, the probe comprises at least 20 nucleotides of the P2RX3sv1 sequence that corresponds to an exon junction polynucleotide created by the alternative splicing of exon 9 to exon 11 of the primary transcript of the P2RX3 gene (see FIGS. 1A and 1B). For example, the polynucleotide sequence: 5′ ATACGGGAATGGAACTGTTC 3′ [SEQ ID NO 16] represents one embodiment of such an inventive P2RX3sv1 polynucleotide wherein a first 10 nucleotide region is complementary and hybridizable to the 3′ end of exon 9 of the P2RX3 gene and a second 10 nucleotide region is complementary and hybridizable to the 5′ end of exon 11 of the P2RX3 gene (see FIG. 1B).

In another embodiment, for detection of P2RX3sv2, the probe comprises at least 20 nucleotides of the P2RX3sv2 sequence that corresponds to an exon junction polynucleotide created by the alternative splicing of exon 10 to intron 10′ of the primary transcript of the P2RX3 gene (see FIGS. 1A and 1B). For example, the polynucleotide sequence: 5′ TGTGGGAGTGGTGCTTCAGT 3′ [SEQ ID NO 17] represents one embodiment of such an inventive P2RX3sv2 polynucleotide wherein a first 10 nucleotide region is complementary and hybridizable to the 3′ end of exon 10 of the P2RX3 gene and a second 10 nucleotide region is complementary and hybridizable to the 5′ end of intron 10′ of the P2RX3 gene (see FIG. 1B).

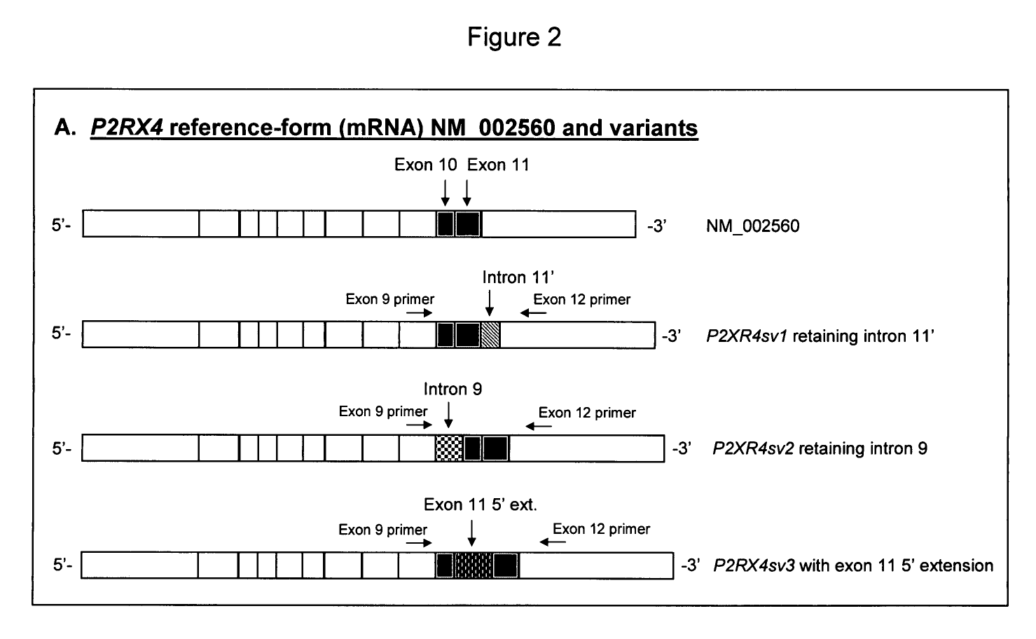

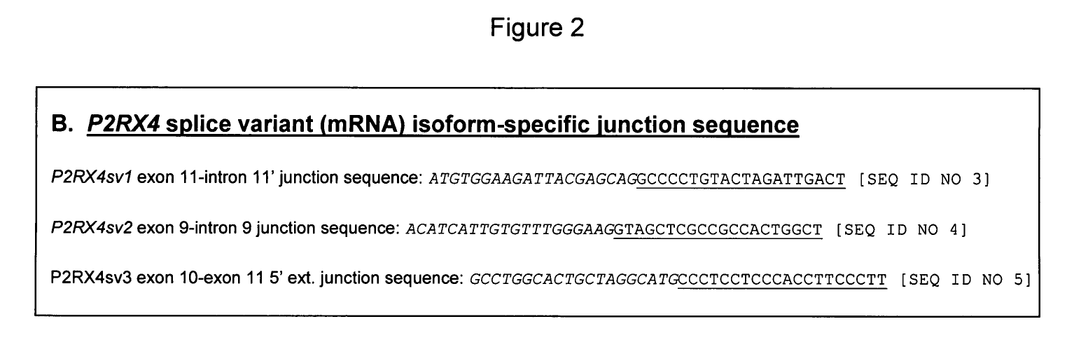

In another embodiment, for detection of P2RX4sv1, the probe comprises at least 20 nucleotides of the P2RX4sv1 sequence that corresponds to an exon junction polynucleotide created by the alternative splicing of exon 11 to intron 11′ of the primary transcript of the P2RX4 gene (see FIGS. 2A and 2B). For example, the polynucleotide sequence: 5′ TTACGAGCAGGCCCCTGTAC 3′ [SEQ ID NO 18] represents one embodiment of such an inventive P2RX4sv1 polynucleotide wherein a first 10 nucleotide region is complementary and hybridizable to the 3′ end of exon 11 of the P2RX4 gene and a second 10 nucleotide region is complementary and hybridizable to the 5′ end of intron 11A of the P2RX4 gene (see FIG. 2B).

In another embodiment, for detection of P2RX4sv2, the probe comprises at least 20 nucleotides of the P2RX4sv2 sequence that corresponds to an exon junction polynucleotide created by the alternative splicing of exon 9 to intron 9 of the primary transcript of the P2RX4 gene (see FIGS. 2A and 2B). For example, the polynucleotide sequence: 5′ GTTTGGGAAGGTAGCTCGCC 3′ [SEQ ID NO 19] represents one embodiment of such an inventive P2RX4sv2 polynucleotide wherein a first 10 nucleotide region is complementary and hybridizable to the 3′ end of exon 9 of the P2RX4 gene and a second 10 nucleotide region is complementary and hybridizable to the 5′ end of intron 9 of the P2RX4 gene (see FIG. 2B).

In another embodiment, for detection of P2RX4sv3, the probe comprises at least 20 nucleotides of the P2RX4sv3 sequence that corresponds to an exon junction polynucleotide created by the alternative splicing of exon 10 to exon 11 5′ extension of the primary transcript of the P2RX4 gene (see FIGS. 2A and 2B). For example, the polynucleotide sequence: 5′ GCTAGGCATGCCCTCCTCCC 3′ [SEQ ID NO 20] represents one embodiment of such an inventive P2RX4sv3 polynucleotide wherein a first 10 nucleotide region is complementary and hybridizable to the 3′ end of exon 10 of the P2RX4 gene and a second 10 nucleotide region is complementary and hybridizable to the 5′ end of exon 11 5′ extension of the P2RX4 gene (see FIG. 2B).

In some embodiments, the first 20 nucleotides of a P2RX3sv1 probe comprise a first continuous region of 5 to 15 nucleotides that is complementary and hybridizable to the 3′ end of exon 9 and a second continuous region of 5 to 15 nucleotides that is complementary and hybridizable to the 5′ end of exon 11. In some embodiments, the first 20 nucleotides of a P2RX3sv2 probe comprise a first continuous region of 5 to 15 nucleotides that is complementary and hybridizable to the 3′ end of exon 10 and a second continuous region of 5 to 15 nucleotides that is complementary and hybridizable to the 5′ end of intron 10′. In some embodiments, the first 20 nucleotides of a P2RX4sv1 probe comprise a first continuous region of 5 to 15 nucleotides that is complementary and hybridizable to the 3′ end of exon 11 and a second continuous region of 5 to 15 nucleotides that is complementary and hybridizable to the 5′ end of intron 11′. In some embodiments, the first 20 nucleotides of a P2RX4sv2 probe comprise a first continuous region of 5 to 15 nucleotides that is complementary and hybridizable to the 3′ end of exon 9 and a second continuous region of 5 to 15 nucleotides that is complementary and hybridizable to the 5′ end of intron 9. In some embodiments, the first 20 nucleotides of a P2RX4sv3 probe comprise a first continuous region of 5 to 15 nucleotides that is complementary and hybridizable to the 3′ end of exon 10 and a second continuous region of 5 to 15 nucleotides that is complementary and hybridizable to the 5′ end of exon 11 5′ extension.

In other embodiments, the P2RX3sv1 or P2RX3sv2 polynucleotide comprises at least 40, 60, 80 or 100 nucleotides of the P2RX3sv1 or P2RX3sv2 sequence, respectively, that correspond to a junction polynucleotide region created by the alternative splicing of exon 9 to exon 11 in the case of P2RX3sv1, or in the case of P2RX3sv2, by the alternative splicing of exon 10 to intron 10′ of the primary transcript of the P2RX3 gene. In embodiments involving P2RX3sv1, the P2RX3sv1 polynucleotide is selected to comprise a first continuous region of at least 5 to 15 nucleotides that is complementary and hybridizable to the 3′ end of exon 9 and a second continuous region of at least 5 to 15 nucleotides that is complementary and hybridizable to the 5′ end of exon 11. Similarly, in embodiments involving P2RX3sv2, the P2RX3sv2 polynucleotide is selected to comprise a first continuous region of at least 5 to 15 nucleotides that is complementary and hybridizable to the 3′ end of exon 10 and a second continuous region of at least 5 to 15 nucleotides that is complementary and hybridizable to the 5′ end of intron 10′. As will be apparent to a person of skill in the art, a large number of different polynucleotide sequences from the region of the exon 9 to exon 11 splice junction and the exon 10 to intron 10′ splice junction may be selected which will, under appropriate hybridization conditions, have the capacity to detectably hybridize to P2RX3sv1 or P2RX3sv2 polynucleotides, respectively, and yet will hybridize to a much less extent or not at all to P2RX3 isoform polynucleotides wherein exon 9 is not spliced to exon 11 or wherein exon 10 is not spliced to intron 10′, respectively.

In other embodiments, the P2RX4sv1, P2RX4sv2, or P2RX4sv3 polynucleotide comprises at least 40, 60, 80 or 100 nucleotides of the P2RX4sv1, P2RX4sv2, or P2RX4sv3 sequence, respectively, that correspond to a junction polynucleotide region created by the alternative splicing of exon 11 to intron 11′ in the case of P2RX4sv1, by the alternative splicing of exon 9 to intron 9 in the case of P2RX4sv2, or in the case of P2RX4sv3, by the alternative splicing of exon 10 to exon 11 5′ extension of the primary transcript of the P2RX4 gene. In embodiments involving P2RX4sv1, the P2RX4sv1 polynucleotide is selected to comprise a first continuous region of at least 5 to 15 nucleotides that is complementary and hybridizable to the 3′ end of exon 11 and a second continuous region of at least 5 to 15 nucleotides that is complementary and hybridizable to the 5′ end of intron 11′. Similarly, in embodiments involving P2RX4sv2, the P2RX4sv2 polynucleotide is selected to comprise a first continuous region of at least 5 to 15 nucleotides that is complementary and hybridizable to the 3′ end of exon 9 and a second continuous region of at least 5 to 15 nucleotides that is complementary and hybridizable to the 5′ end of intron 9. Similarly, in embodiments involving P2RX4sv3, the P2RX4sv3 polynucleotide is selected to comprise a first continuous region of at least 5 to 15 nucleotides that is complementary and hybridizable to the 3′ end of exon 10 and a second continuous region of at least 5 to 15 nucleotides that is complementary and hybridizable to the 5′ end of exon 11 5′ extension. As will be apparent to a person of skill in the art, a large number of different polynucleotide sequences from the region of the exon 11 to intron 11′ splice junction, exon 9 to intron 9, and the exon 10 to exon 11 5′ extension splice junction may be selected which will, under appropriate hybridization conditions, have the capacity to detectably hybridize to P2RX4sv1, P2RX4sv2, or P2RX4sv3 polynucleotides, respectively, and yet will hybridize to a much less extent or not at all to P2RX4 isoform polynucleotides wherein exon 11 is not spliced to intron 11′, exon 9 is not spliced to intron 9, or wherein exon 10 is not spliced to exon 11 5′ extension, respectively.

Preferably, non-complementary nucleic acid that is present has a particular purpose such as being a reporter sequence or being a capture sequence. However, additional nucleic acid need not have a particular purpose as long as the additional nucleic acid does not prevent the P2RX3sv1 or P2RX3sv2 nucleic acid from distinguishing between target polynucleotides, e.g., P2RX3sv1 or P2RX3sv2 polynucleotides, and non-target polynucleotides, including, but not limited to P2RX3 polynucleotides not comprising the exon 9 to exon 11 splice junction or the exon 10 to intron 10′ splice junctions found in P2RX3sv1 or P2RX3sv2, respectively. Similarly, additional nucleic acid need not have a particular purpose as long as the additional nucleic acid does not prevent the P2RX4sv1, P2RX4sv2, or P2RX4sv3 nucleic acid from distinguishing between target polynucleotides, e.g., P2RX4sv1, P2RX4sv2, or P2RX4sv3 polynucleotides, and non-target polynucleotides, including, but not limited to P2RX4 polynucleotides not comprising the exon 11 to intron 11′ splice junction, the exon 9 to intron 9 splice junction, or the exon 10 to exon 11 5′ extension splice junctions found in P2RX4sv1, P2RX4sv2, or P2RX4sv3, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

P2RX3sv1, P2RX3sv2, P2RX4sv1, P2RX4sv2, or P2RX4sv3 polynucleotides, such as those comprising SEQ ID NO 6, SEQ ID NO 8, SEQ ID NO 10, SEQ ID NO 12, or SEQ ID NO 14, respectively, can be used to make P2RX3sv1, P2RX3sv2, P2RX4sv1, P2RX4sv2, or P2RX4sv3 polypeptides, respectively. In particular, P2RX3sv1, P2RX3sv2, P2RX4sv1, P2RX4sv2, or P2RX4sv3 polypeptides can be expressed from recombinant nucleic acids in a suitable host or in vitro using a translation system. Recombinantly expressed P2RX3sv1, P2RX3sv2, P2RX4sv1, P2RX4sv2, or P2RX4sv3 polypeptides can be used, for example, in assays to screen for compounds that bind P2RX3sv1, P2RX3sv2, P2RX4sv1, P2RX4sv2, or P2RX4sv3, respectively. Alternatively, P2RX3sv1 or P2RX3sv2 polypeptides can also be used to screen for compounds that bind to one or more P2RX3 or purinergic receptor P2X isoforms, but do not bind to P2RX3sv1 or P2RX3sv2, respectively. Alternatively, P2RX4sv1, P2RX4sv2, or P2RX4sv3 polypeptides can also be used to screen for compounds that bind to one or more P2RX4 or purinergic receptor P2X isoforms, but do not bind to P2RX4sv1, P2RX4sv2, or P2RX4sv3, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMClneo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 6, SEQ ID NO 8, SEQ ID NO 10, SEQ ID NO 12, or SEQ ID NO 14 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

P2RX3sv1, P2RX3sv2, P2RX4sv1, P2RX4sv2, and P2RX4sv3 Polypeptides

P2RX3sv1 polypeptides contain an amino acid sequence comprising, consisting or consisting essentially of SEQ ID NO 7. P2RX3sv2 polypeptides contain an amino acid sequence comprising, consisting or consisting essentially of SEQ ID NO 9. P2RX4sv1 polypeptides contain an amino acid sequence comprising, consisting or consisting essentially of SEQ ID NO 11. P2RX4sv2 polypeptides contain an amino acid sequence comprising, consisting or consisting essentially of SEQ ID NO 13. P2RX4sv3 polypeptides contain an amino acid sequence comprising, consisting or consisting essentially of SEQ ID NO 15. P2RX3sv1 or P2RX3sv2 polypeptides have a variety of uses, such as providing a marker for the presence of P2RX3sv1 or P2RX3sv2, respectively; use as an immunogen to produce antibodies binding to P2RX3sv1 or P2RX3sv2, respectively; use as a target to identify compounds binding selectively to P2RX3sv1 or P2RX3sv2, respectively; or use in an assay to identify compounds that bind to one or more P2RX3 or purinergic receptor P2X isoforms but do not bind to or interact with P2RX3sv1 or P2RX3sv2, respectively. Similarly, P2RX4sv1, P2RX4sv2, or P2RX4sv3 polypeptides have a variety of uses, such as providing a marker for the presence of P2RX4sv1, P2RX4sv2, or P2RX4sv3, respectively; use as an immunogen to produce antibodies binding to P2RX4sv1, P2RX4sv2, or P2RX4sv3, respectively; use as a target to identify compounds binding selectively to P2RX4sv1, P2RX4sv2, or P2RX4sv3, respectively; or use in an assay to identify compounds that bind to one or more P2RX4 or purinergic receptor P2X isoforms but do not bind to or interact with P2RX4sv1, P2RX4sv2, or P2RX4sv3, respectively.

In chimeric polypeptides containing one or more regions from P2RX3sv1, P2RX3sv2, P2RX4sv1, P2RX4sv2, or P2RX4sv3 and one or more regions not from P2RX3sv1, P2RX3sv2, P2RX4sv1, P2RX4sv2, or P2RX4sv3, respectively, the region(s) not from P2RX3sv1, P2RX3sv2, P2RX4sv1, P2RX4sv2, or P2RX4sv3, respectively, can be used, for example, to achieve a particular purpose or to produce a polypeptide that can substitute for P2RX3sv1, P2RX3sv2, P2RX4sv1, P2RX4sv2, or P2RX4sv3, or fragments thereof. Particular purposes that can be achieved using chimeric P2RX3sv1 or P2RX3sv2 polypeptides include providing a marker for P2RX3sv1 or P2RX3sv2 activity, respectively, and altering the activity and regulation of the P2RX3 ligand gated ion channel. Particular purposes that can be achieved using chimeric P2RX4sv1, P2RX4sv2, or P2RX4sv3 polypeptides include providing a marker for P2RX4sv1, P2RX4sv2, or P2RX4sv3 activity, respectively, and altering the activity and regulation of the P2RX4 ligand gated ion channel.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional P2RX3sv1, P2RX3sv2, P2RX4sv1, P2RX4sv2, and P2RX4sv3

Functional P2RX3sv1 and P2RX3sv2 are different protein isoforms of P2RX3. Functional P2RX4sv1, P2RX4sv2, and P2RX4sv3 are different protein isoforms of P2RX4. The identification of the amino acid and nucleic acid sequences of P2RX3sv1, P2RX3sv2, P2RX4sv1, P2RX4sv2, or P2RX4sv3 provide tools for obtaining functional proteins related to P2RX3sv1, P2RX3sv2, P2RX4sv1, P2RX4sv2, or P2RX4sv3, respectively, from other sources, for producing P2RX3sv1, P2RX3sv2, P2RX4sv1, P2RX4sv2, or P2RX4sv3 chimeric proteins, and for producing functional derivatives of SEQ ID NO 7, SEQ ID NO 9, SEQ ID NO 11, SEQ ID NO 13, or SEQ ID NO 15.

P2RX3sv1, P2RX3sv2, P2RX4sv1, P2RX4sv2, or P2RX4sv3 polypeptides can be readily identified and obtained based on their sequence similarity to P2RX3sv1 (SEQ ID NO 7), P2RX3sv2 (SEQ ID NO 9), P2RX4sv1 (SEQ ID NO 11), P2RX4sv2 (SEQ ID NO 13), or P2RX4sv3 (SEQ ID NO 15), respectively. In particular, P2RX3sv1 lacks the amino acids encoded by exon 10 the P2RX3 gene. The deletion of exon 10 and the splicing of exon 9 to exon 11 of the P2RX3 heteronuclear RNA (hnRNA) transcript do not result in a shift of the protein reading frame at the exon 9 to exon 11 splice junction. Thus, the P2RX3sv1 polypeptide is lacking 22 amino acids encoded by nucleotides corresponding to exon 10 of the P2RX3 hnRNA. The P2RX3sv2 polypeptides contain unique amino acids at the C-terminus, encoded by nucleotides located after the splice junction that results from the retention of a 216 nucleotide portion of intron 10 of the P2RX3 gene. The addition of this portion of intron 10, referred to as intron 10′, does not disrupt the protein reading frame as compared to the P2RX3 reference sequence (NP_{—}002559), but introduces a stop codon 121 nucleotides downstream of the exon 10/intron 10′ splice junction. Therefore, P2RX3sv2 polypeptide contains 40 unique amino acids encoded by nucleotides corresponding to intron 10′ at the C-terminus and is also lacking amino acids encoded by the nucleotides downstream of the premature stop codon compared to the P2RX3 reference sequence (NP_{—}002559). The P2RX4sv1 polypeptides contain unique amino acids, encoded by nucleotides located after the splice junction that results from the retention of a 58 nucleotide portion of intron 11 of the P2RX4 gene. The addition of this portion of intron 11, referred to as intron 11′, does change the protein reading frame as compared to the P2RX4 reference sequence (NP_{—}002560). The insertion also creates a premature termination codon 16 nucleotides downstream of the exon 11/intron 11′ splice junction. Thus, the P2RX4sv1 polypeptide contains 5 unique amino acids at the C-terminus and is also lacking the amino acids encoded by the nucleotides downstream of the premature stop codon. The P2RX4sv2 polypeptides contain unique amino acids, encoded by nucleotides located after the splice junction that results from the retention of intron 9 of the P2RX4 gene. The addition of intron 9 does disrupt the protein reading frame as compared to the P2RX4 reference sequence (NP_{—}002560). The change in protein reading frame creates a premature termination codon 12 nucleotides downstream of the intron 9/exon 10 splice junction. Thus, the P2RX4sv2 polypeptide contains 38 unique amino acids at the C-terminus and is also lacking the amino acids encoded by the nucleotides downstream of the premature stop codon. The P2RX4sv3 polypeptides contain unique amino acids encoded by nucleotides located after the splice junction that results from the retention of a 183 nucleotide portion of intron 10 of the P2RX4 gene, extending the 5′ end of exon 11. The addition of this portion of exon 10, referred to as exon 11 5′ extension, does not change the protein reading frame as compared to the P2RX4 reference sequence (NP_{—}002560). The insertion does create a premature termination codon 175 nucleotides downstream of the exon 10/exon 11 5′ extension splice junction. Thus, the P2RX4sv3 polypeptide contains 58 unique amino acids at the C-terminus and is also lacking the amino acids encoded by the nucleotides downstream of the premature stop codon.

Both the amino acid and nucleic acid sequences of P2RX3sv1, P2RX3sv2, P2RX4sv1, P2RX4sv2, or P2RX4sv3 can be used to help identify and obtain P2RX3sv1, P2RX3sv2, P2RX4sv1, P2RX4sv2, or P2RX4sv3 polypeptides, respectively. For example, SEQ ID NO 6 can be used to produce degenerative nucleic acid probes or primers for identifying and cloning nucleic acid polynucleotides encoding for a P2RX3sv1 polypeptide. In addition, polynucleotides comprising, consisting, or consisting essentially of SEQ ID NO 6 or fragments thereof, can be used under conditions of moderate stringency to identify and clone nucleic acids encoding P2RX3sv1 polypeptides from a variety of different organisms. The same methods can also be performed with polynucleotides comprising, consisting, or consisting essentially of SEQ ID NO 8, SEQ ID NO 10, SEQ ID NO 12, or SEQ ID NO 14, or fragments thereof, to identify and clone nucleic acids encoding P2RX3sv2, P2RX4sv1, P2RX4sv2, and P2RX4sv3, respectively.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with P2RX3sv1, P2RX3sv2, P2RX4sv1, P2RX4sv2, or P2RX4sv3 obtained from a particular source, derivatives can be produced. Such derivatives include polypeptides with amino acid substitutions, additions and deletions. Changes to P2RX3sv1, P2RX3sv2, P2RX4sv1, P2RX4sv2, or P2RX4sv3 to produce a derivative having essentially the same properties should be made in a manner not altering the tertiary structure of P2RX3sv1, P2RX3sv2, P2RX4sv1, P2RX4sv2, or P2RX4sv3, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

P2RX3sv1, P2RX3sv2, P2RX4sv1, P2RX4sv2, and P2RX4sv3 Antibodies

Antibodies recognizing P2RX3sv1, P2RX3sv2, P2RX4sv1, P2RX4sv2, or P2RX4sv3 can be produced using a polypeptide containing SEQ ID NO 7 in the case of P2RX3sv1, SEQ ID NO 9 in the case of P2RX3sv2, SEQ ID NO 11 in the case of P2RX4sv1, SEQ ID NO 13 in the case of P2RX4sv2, or SEQ ID NO 15 in the case of P2RX4sv3, respectively, or a fragment thereof, as an immunogen. Preferably, a P2RX3sv1 polypeptide used as an immunogen consists of a polypeptide of SEQ ID NO 7 or a SEQ ID NO 7 fragment having at least 10 contiguous amino acids in length corresponding to the polynucleotide region representing the junction resulting from the splicing of exon 9 to exon 11 of the P2RX3 gene. Preferably, a P2RX3sv2 polypeptide used as an immunogen consists of a polypeptide derived from SEQ ID NO 9 or a SEQ ID NO 9 fragment, having at least 10 contiguous amino acids in length corresponding to the polynucleotide region representing the junction resulting from the splicing of exon 10 to intron 10′ of the P2RX3 gene. Preferably, a P2RX4sv1 polypeptide used as an immunogen consists of a polypeptide derived from SEQ ID NO 11 or a SEQ ID NO 11 fragment, having at least 10 contiguous amino acids in length corresponding to the polynucleotide region representing the junction resulting from the splicing of exon 10 to intron 11′ of the P2RX4 gene. Preferably, a P2RX4sv2 polypeptide used as an immunogen consists of a polypeptide derived from SEQ ID NO 13 or a SEQ ID NO 13 fragment, having at least 10 contiguous amino acids in length corresponding to the polynucleotide region representing the junction resulting from the splicing of exon 9 to intron 9 of the P2RX4 gene. Preferably, a P2RX4sv3 polypeptide used as an immunogen consists of a polypeptide derived from SEQ ID NO 15 or a SEQ ID NO 15 fragment, having at least 10 contiguous amino acids in length corresponding to the polynucleotide region representing the junction resulting from the splicing of exon 10 to exon 11 5′ extension of the P2RX4 gene.

In some embodiments where, for example, P2RX3sv1 polypeptides are used to develop antibodies that bind specifically to P2RX3sv1 and not to other isoforms of P2RX3, the P2RX3sv1 polypeptides comprise at least 10 amino acids of the P2RX3sv1 polypeptide sequence corresponding to a junction polynucleotide region created by the alternative splicing of exon 9 to exon 11 of the primary transcript of the P2RX3 gene (see FIG. 1). For example, the amino acid sequence: amino terminus-LVYGNGTVLC-carboxy terminus [SEQ ID NO 21] represents one embodiment of such an inventive P2RX3sv1 polypeptide wherein a first 5 amino acid region is encoded by nucleotide sequence at the 3′ end of exon 9 of the P2RX3 gene and a second 5 amino acid region is encoded by the nucleotide sequence at the 5′ end of exon 11 of the P2RX3 gene. Preferably, at least 10 amino acids of the P2RX3sv1 polypeptide comprise a first continuous region of 1 to 8 amino acids that is encoded by nucleotides at the 3′ end of exon 9 and a second continuous region of 1 to 8 amino acids that is encoded by nucleotides at the 5′ end of exon 11.

In other embodiments where, for example, P2RX3sv2 polypeptides are used to develop antibodies that bind specifically to P2RX3sv2 and not to other isoforms of P2RX3, the P2RX3sv2 polypeptides comprise at least 10 amino acids of the P2RX3sv2 sequence corresponding to a junction polynucleotide region created by the alternative splicing of exon 10 to intron 10′ of the primary transcript of the P2RX3 gene (see FIG. 1). For example, the amino acid sequence: amino terminus-TSVGVVLQSP-carboxy terminus [SEQ ID NO 22], represents one embodiment of such an inventive P2RX3sv2 polypeptide wherein a first 5 amino acid region is encoded by nucleotide sequence at the 3′ end of exon 10 of the P2RX3 gene and a second 5 amino acid region is encoded by the nucleotide sequence at the 5′ end of intron 10′ of the P2RX3 gene. Preferably, at least 10 amino acids of the P2RX3sv2 polypeptide comprise a first continuous region of 1 to 8 amino acids that is encoded by nucleotides at the 3′ end of exon 10 and a second continuous region of 1 to 8 amino acids that is encoded by nucleotides at the 5′ end of intron 10′.

In other embodiments where, for example, P2RX4sv1 polypeptides are used to develop antibodies that bind specifically to P2RX4sv1 and not to other P2RX4 isoforms, the P2RX4sv1 polypeptides comprise at least 10 amino acids of the P2RX4sv1 polypeptide sequence corresponding to a junction polynucleotide region created by the alternative splicing of exon 11 to intron 11′ of the primary transcript of the P2RX4 gene (see FIG. 2). For example, the amino acid sequence: amino terminus-EDYEQAPVLD-carboxy terminus [SEQ ID NO 23], represents one embodiment of such an inventive P2RX4sv1 polypeptide wherein a first 5 amino acid region is encoded by a nucleotide sequence at the 5′ end of exon 11 of the P2RX4 gene and a second 5′ amino acid region is encoded by the nucleotide sequence at the 5′ end of intron 11′ of the P2RX4 gene. Preferably, at least 10 amino acids of the P2RX4sv1 polypeptide comprise a first continuous region of 1 to 8 amino acids that is encoded by nucleotides at the 3′ end of exon 11 and a second continuous region of 1 to 8 amino acids that is encoded by nucleotides at the 5′ end of intron 11′.

In other embodiments where, for example, P2RX4sv2 polypeptides are used to develop antibodies that bind specifically to P2RX4sv2 and not to other P2RX4 isoforms, the P2RX4sv2 polypeptides comprise at least 10 amino acids of the P2RX4sv2 polypeptide sequence corresponding to a junction polynucleotide region created by the alternative splicing of exon 9 to intron 9 of the primary transcript of the P2RX4 gene (see FIG. 2). For example, the amino acid sequence: amino terminus-IVFGKVARRH-carboxy terminus [SEQ ID NO 24], represents one embodiment of such an inventive P2RX4sv2 polypeptide wherein a first 5 amino acid region is encoded by a nucleotide sequence at the 5′ end of exon 9 of the P2RX4 gene and a second 5′ amino acid region is encoded by the nucleotide sequence at the 5′ end of intron 9 of the P2RX4 gene. Preferably, at least 10 amino acids of the P2RX4sv2 polypeptide comprise a first continuous region of 1 to 8 amino acids that is encoded by nucleotides at the 3′ end of exon 9 and a second continuous region of 1 to 8 amino acids that is encoded by nucleotides at the 5′ end of intron 9.

In other embodiments where, for example, P2RX4sv3 polypeptides are used to develop antibodies that bind specifically to P2RX4sv3 and not to other P2RX4 isoforms, the P2RX4sv3 polypeptides comprise at least 10 amino acids of the P2RX4sv3 polypeptide sequence corresponding to a junction polynucleotide region created by the alternative splicing of exon 10 to exon 11 5′ extension of the primary transcript of the P2RX4 gene (see FIG. 2). For example, the amino acid sequence: amino terminus-ALLGMPSSHL-carboxy terminus [SEQ ID NO 25], represents one embodiment of such an inventive P2RX4sv3 polypeptide wherein a first 5 amino acid region is encoded by a nucleotide sequence at the 5′ end of exon 10 of the P2RX4 gene and a second 5′ amino acid region is encoded by the nucleotide sequence at the 5′ end of exon 11 5′ extension of the P2RX4 gene. Preferably, at least 10 amino acids of the P2RX4sv3 polypeptide comprise a first continuous region of 1 to 8 amino acids that is encoded by nucleotides at the 3′ end of exon 10 and a second continuous region of 1 to 8 amino acids that is encoded by nucleotides at the 5′ end of exon 11 5′ extension.

In other embodiments, P2RX3sv1-specific antibodies are made using a P2RX3sv1 polypeptide that comprises at least 20, 30, 40 or 50 amino acids of the P2RX3sv1 sequence that corresponds to a junction polynucleotide region created by the alternative splicing of exon 9 to exon 11 of the primary transcript of the P2RX3 gene. In each case the P2RX3sv1 polypeptides are selected to comprise a first continuous region of at least 5 to 15 amino acids that is encoded by nucleotides at the 3′ end of exon 9 and a second continuous region of 5 to 15 amino acids that is encoded by nucleotides directly after the novel splice junction.

In other embodiments, P2RX3sv2-specific antibodies are made using a P2RX3sv2 polypeptide that comprises at least 20, 30, 40 or 50 amino acids of the P2RX3sv2 sequence that corresponds to a junction polynucleotide region created by the alternative splicing of exon 10 to intron 10′ of the primary transcript of the P2RX3 gene. In each case the P2RX3sv1 polypeptides are selected to comprise a first continuous region of at least 5 to 15 amino acids that is encoded by nucleotides at the 3′ end of exon 10 and a second continuous region of 5 to 15 amino acids that is encoded by nucleotides directly after the novel splice junction.

In other embodiments, P2RX4sv1-specific antibodies are made using a P2RX4sv1 polypeptide that comprises at least 20, 30, 40 or 50 amino acids of the P2RX4sv1 sequence that corresponds to a junction polynucleotide region created by the alternative splicing of exon 11 to intron 11′ of the primary transcript of the P2RX4 gene. In each case the P2RX4sv1 polypeptides are selected to comprise a first continuous region of at least 5 to 15 amino acids that is encoded by nucleotides at the 3′ end of exon 11 and a second continuous region of 5 to 15 amino acids that is encoded by nucleotides directly after the novel splice junction.

In other embodiments, P2RX4sv2-specific antibodies are made using a P2RX4sv2 polypeptide that comprises at least 20, 30, 40 or 50 amino acids of the P2RX4sv2 sequence that corresponds to a junction polynucleotide region created by the alternative splicing of exon 9 to intron 9 of the primary transcript of the P2RX4 gene. In each case the P2RX4sv2 polypeptides are selected to comprise a first continuous region of at least 5 to 15 amino acids that is encoded by nucleotides at the 3′ end of exon 9 and a second continuous region of 5 to 15 amino acids that is encoded by nucleotides directly after the novel splice junction.

In other embodiments, P2RX4sv3-specific antibodies are made using a P2RX4sv3 polypeptide that comprises at least 20, 30, 40 or 50 amino acids of the P2RX4sv3 sequence that corresponds to a junction polynucleotide region created by the alternative splicing of exon 10 to exon 11 5′ extension of the primary transcript of the P2RX4 gene. In each case the P2RX4sv3 polypeptides are selected to comprise a first continuous region of at least 5 to 15 amino acids that is encoded by nucleotides at the 3′ end of exon 10 and a second continuous region of 5 to 15 amino acids that is encoded by nucleotides directly after the novel splice junction.

Antibodies to P2RX3sv1, P2RX3sv2, P2RX4sv1, P2RX4sv2, or P2RX4sv3 have different uses, such as to identify the presence of P2RX3sv1, P2RX3sv2, P2RX4sv1, P2RX4sv2, or P2RX4sv3, respectively, and to isolate P2RX3sv1, P2RX3sv2, P2RX4sv1, P2RX4sv2, or P2RX4sv3 polypeptides, respectively. Identifying the presence of P2RX3sv1 can be used, for example, to identify cells producing P2RX3sv1. Such identification provides an additional source of P2RX3sv1 and can be used to distinguish cells known to produce P2RX3sv1 from cells that do not produce P2RX3sv1. For example, antibodies to P2RX3sv1 can distinguish human cells expressing P2RX3sv1 from human cells not expressing P2RX3sv1 or non-human cells (including bacteria) that do not express P2RX3sv1. Such P2RX3sv1 antibodies can also be used to determine the effectiveness of P2RX3sv1 ligands, using techniques well known in the art, to detect and quantify changes in the protein levels of P2RX3sv1 in cellular extracts, and in situ immunostaining of cells and tissues. In addition, the same above-described utilities also exist for P2RX3sv2-specific antibodies, P2RX4sv1-specific antibodies, P2RX4sv2-specific antibodies, and P2RX4sv3-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

P2RX3sv1, P2RX3sv2, P2RX4sv1, P2RX4sv2, and P2RX4sv3 Binding Assay

A number of compounds known to modulate purinergic receptor P2X activity have been disclosed including suramin, PPADS, Zn²⁺, and A-317491 (reviewed in North, 2002, Physiol. Rev. 82:1013-1067; Lambrecht, 2000, Naunyn-Schmiedeberg's Arch. Pharmacol. 362:340-350; Jarvis et al., 2002, Proc. Natl. Acad. Sci. USA 99:17179-17184). Fluorenes and anthracenes that inhibit P2RX3 have also been disclosed (U.S. Pat. No. 6,693,136). A splice variant of the mouse P2RX4 exhibits channel properties compared to the reference mouse protein and affects the function of heteromeric P2RX4 channels composed of the two, indicating that splice variant isoforms of purinergic receptors P2X may have different sensitivities to ligands (Townsend-Nicholson et al., 1999, Mol Brain Res. 64:246-254). Methods for expressing purinergic receptors P2X in Xenopus oocytes and monitoring the activity of these channels, including analyzing the effect of compounds on the activity of purinergic receptor P2X ligand-gated ion channel activity, have been described previously (U.S. Pat. No. 6,214,581; U.S. Pat. No. 6,242,216; Xiong et al., 2000, Br. J. Pharmacol. 130:1394-1398; Garcia-Guzman et al., 1997, Mol. Brain Res. 47:59-66). Methods and compositions useful in the identification of compounds affecting ligand-gated channel activity have also been described (see for example US 2004/0132187). A person skilled in the art may use these methods to screen P2RX3sv1 or P2RX3sv2 polypeptides for compounds that bind to, and in some cases functionally alter, each respective P2RX3 isoform protein. A person skilled in the art should also be able to use these methods to screen P2RX4sv1, P2RX4sv2, or P2RX4sv3 polypeptides for compounds that bind to, and in some cases functionally alter, each respective P2RX4 isoform protein.

P2RX3sv1, P2RX3sv2, P2RX4sv1, P2RX4sv2, P2RX4sv3, or fragments thereof, can be used in binding studies to identify compounds binding to or interacting with P2RX3sv1, P2RX3sv2, P2RX4sv1, P2RX4sv2, P2RX4sv3, or fragments thereof, respectively. In one embodiment, P2RX3sv1, or a fragment thereof, can be used in binding studies with a purinergic receptor P2X isoform protein, or a fragment thereof, to identify compounds that: bind to or interact with P2RX3sv1 and other purinergic receptor P2X isoforms; bind to or interact with one or more other purinergic receptor P2X isoforms and not with P2RX3sv1; bind to or interact with P2RX3sv1 and not with one or more other purinergic receptor P2X isoforms. A similar series of compound screens can, of course, also be performed using P2RX3sv2, P2RX4sv1, P2RX4sv2, or P2RX4sv3 rather than, or in addition to, P2RX3sv1. Such binding studies can be performed using different formats including competitive and non-competitive formats. Further competition studies can be carried out using additional compounds determined to bind to P2RX3sv1, P2RX3sv2, P2RX4sv1, P2RX4sv2, P2RX4sv3, other P2RX3 isoforms, other P2RX4 isoforms, or other purinergic receptor P2X isoforms.

The particular P2RX3sv1, P2RX3sv2, P2RX4sv1, P2RX4sv2, or P2RX4sv3 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using P2RX3sv1 expressed from a recombinant nucleic acid. Alternatively, recombinantly expressed P2RX3sv1 consists of the SEQ ID NO 7 amino acid sequence. In addition, binding studies are performed using P2RX3sv2 expressed from a recombinant nucleic acid. Alternatively, recombinantly expressed P2RX3sv2 consists of the SEQ ID NO 9 amino acid sequence. In addition, binding studies are performed using P2RX4sv1 expressed from a recombinant nucleic acid. Alternatively, recombinantly expressed P2RX4sv1 consists of the SEQ ID NO 11 amino acid sequence. In addition, binding studies are performed using P2RX4sv2 expressed from a recombinant nucleic acid. Alternatively, recombinantly expressed P2RX4sv2 consists of the SEQ ID NO 13 amino acid sequence. In addition, binding studies are performed using P2RX4sv3 expressed from a recombinant nucleic acid. Alternatively, recombinantly expressed P2RX4sv3 consists of the SEQ ID NO 15 amino acid sequence.

Binding assays can be performed using individual compounds or preparations containing different numbers of compounds. A preparation containing different numbers of compounds having the ability to bind to P2RX3sv1, P2RX3sv2, P2RX4sv1, P2RX4sv2, or P2RX4sv3 can be divided into smaller groups of compounds that can be tested to identify the compound(s) binding to P2RX3sv1, P2RX3sv2, P2RX4sv1, P2RX4sv2, or P2RX4sv3, respectively.

Binding assays can be performed using recombinantly produced P2RX3sv1, P2RX3sv2, P2RX4sv1, P2RX4sv2, or P2RX4sv3 present in different environments. Such environments include, for example, cell extracts and purified cell extracts containing a P2RX3sv1, P2RX3sv2, P2RX4sv1, P2RX4sv2, or P2RX4sv3 recombinant nucleic acid; and also include, for example, the use of a purified P2RX3sv1, P2RX3sv2, P2RX4sv1, P2RX4sv2, or P2RX4sv3 polypeptide produced by recombinant means which is introduced into different environments.

In one embodiment of the invention, a binding method is provided for screening for a compound able to bind selectively to P2RX3sv1. The method comprises the steps: providing a P2RX3sv1 polypeptide comprising SEQ ID NO 7; providing a purinergic receptor P2X isoform polypeptide that is not P2RX3sv1; contacting the P2RX3sv1 polypeptide and the purinergic receptor P2X isoform polypeptide that is not P2RX3sv1 with a test preparation comprising one or more test compounds; and then determining the binding of the test preparation to the P2RX3sv1 polypeptide and to the purinergic receptor P2X isoform polypeptide that is not P2RX3sv1, wherein a test preparation that binds to the P2RX3sv1 polypeptide, but does not bind to the purinergic receptor P2X isoform polypeptide that is not P2RX3sv1, contains one or more compounds that selectively bind to P2RX3sv1.

In one embodiment of the invention, a binding method is provided for screening for a compound able to bind selectively to P2RX3sv2. The method comprises the steps: providing a P2RX3sv2 polypeptide comprising SEQ ID NO 9; providing a purinergic receptor P2X isoform polypeptide that is not P2RX3sv2; contacting the P2RX3sv2 polypeptide and the purinergic receptor P2X isoform polypeptide that is not P2RX3sv2 with a test preparation comprising one or more test compounds; and then determining the binding of the test preparation to the P2RX3sv2 polypeptide and to the purinergic receptor P2X isoform polypeptide that is not P2RX3sv2, wherein a test preparation that binds to the P2RX3sv2 polypeptide, but does not bind to the purinergic receptor P2X isoform polypeptide that is not P2RX3sv2, contains one or more compounds that selectively bind to P2RX3sv2.

In another embodiment of the invention, a binding method is provided for screening for a compound able to bind selectively to P2RX4sv1. The method comprises the steps: providing a P2RX4sv1 polypeptide comprising SEQ ID NO 11; providing a purinergic receptor P2X isoform polypeptide that is not P2RX4sv1; contacting the P2RX4sv1 polypeptide and the purinergic receptor P2X isoform polypeptide that is not P2RX4sv1 with a test preparation comprising one or more test compounds; and then determining the binding of the test preparation to the P2RX4sv1 polypeptide and to the purinergic receptor P2X isoform polypeptide that is not P2RX4sv1, wherein a test preparation that binds to the P2RX4sv1 polypeptide, but does not bind to the purinergic receptor P2X isoform polypeptide that is not P2RX4sv1, contains one or more compounds that selectively bind to P2RX4sv1.

In another embodiment of the invention, a binding method is provided for screening for a compound able to bind selectively to P2RX4sv2. The method comprises the steps: providing a P2RX4sv2 polypeptide comprising SEQ ID NO 13; providing a purinergic receptor P2X isoform polypeptide that is not P2RX4sv2; contacting the P2RX4sv2 polypeptide and the purinergic receptor P2X isoform polypeptide that is not P2RX4sv2 with a test preparation comprising one or more test compounds; and then determining the binding of the test preparation to the P2RX4sv2 polypeptide and to the purinergic receptor P2X isoform polypeptide that is not P2RX4sv2, wherein a test preparation that binds to the P2RX4sv2 polypeptide, but does not bind to the purinergic receptor P2X isoform polypeptide that is not P2RX4sv2, contains one or more compounds that selectively bind to P2RX4sv2.

In another embodiment of the invention, a binding method is provided for screening for a compound able to bind selectively to P2RX4sv3. The method comprises the steps: providing a P2RX4sv3 polypeptide comprising SEQ ID NO 15; providing a purinergic receptor P2X isoform polypeptide that is not P2RX4sv3; contacting the P2RX4sv3 polypeptide and the purinergic receptor P2X isoform polypeptide that is not P2RX4sv3 with a test preparation comprising one or more test compounds; and then determining the binding of the test preparation to the P2RX4sv3 polypeptide and to the purinergic receptor P2X isoform polypeptide that is not P2RX4sv3, wherein a test preparation that binds to the P2RX4sv3 polypeptide, but does not bind to the purinergic receptor P2X isoform polypeptide that is not P2RX4sv3, contains one or more compounds that selectively bind to P2RX4sv3.

In another embodiment of the invention, a binding method is provided for screening for a compound able to bind selectively to a purinergic receptor P2X isoform polypeptide that is not P2RX3sv1. The method comprises the steps: providing a P2RX3sv1 polypeptide comprising SEQ ID NO 7; providing a purinergic receptor P2X isoform polypeptide that is not P2RX3sv1; contacting the P2RX3sv1 polypeptide and the purinergic receptor P2X isoform polypeptide that is not P2RX3sv1 with a test preparation comprising one or more test compounds; and then determining the binding of the test preparation to the P2RX3sv1 polypeptide and the purinergic receptor P2X isoform polypeptide that is not P2RX3sv1, wherein a test preparation that binds the purinergic receptor P2X isoform polypeptide that is not P2RX3sv1, but does not bind P2RX3sv1, contains a compound that selectively binds the purinergic receptor P2X isoform polypeptide that is not P2RX3sv1. Alternatively, the above method can be used to identify compounds that bind selectively to a purinergic receptor P2X polypeptide that is not P2RX3sv2 by performing the method with P2RX3sv2 protein comprising SEQ ID NO 9. Alternatively, the above method can be used to identify compounds that bind selectively to a purinergic receptor P2X isoform polypeptide that is not P2RX4sv1 by performing the method with P2RX4sv1 protein comprising SEQ ID NO 11. Alternatively, the above method can be used to identify compounds that bind selectively to a purinergic receptor P2X isoform polypeptide that is not P2RX4sv2 by performing the method with P2RX4sv2 protein comprising SEQ ID NO 13. Alternatively, the above method can be used to identify compounds that bind selectively to a purinergic receptor P2X isoform polypeptide that is not P2RX4sv3 by performing the method with P2RX4sv3 protein comprising SEQ ID NO 15.

The above-described selective binding assays can also be performed with a polypeptide fragment of P2RX3sv1 or P2RX3sv2, wherein the polypeptide fragment comprises at least 10 consecutive amino acids that are coded by a nucleotide sequence that bridges the junction created by the splicing of the 3′ end of exon 9 to the 5′ end of exon 11 in the case of P2RX3sv1 or by a nucleotide sequence that bridges the junction created by the splicing of the 3′ end of exon 10 to the 5′ end of intron 10′ or the 3′ end of intron 10′ to the 5′ end of exon 11 in the case of P2RX3sv2. Similarly, the selective binding assays may also be performed using a polypeptide fragment of a purinergic receptor P2X isoform polypeptide that is not P2RX3sv1 or P2RX3sv2, wherein the polypeptide fragment comprises at least 10 consecutive amino acids that are coded by: a) a nucleotide sequence that is contained within exon 10 of the P2RX3 gene; or b) a nucleotide sequence that bridges the junction created by the splicing of the 3′ end of exon 9 to the 5′ end of exon 10, the splicing of the 3′ end of exon 10 to the 5′ end of exon 11, or the splicing of the 3′ end of exon 10 to the 5′ end of exon 11 of the P2RX3 gene.

The above-described selective binding assays can also be performed with a polypeptide fragment of P2RX4sv1, P2RX4sv2, or P2RX4sv3, wherein the polypeptide fragment comprises at least 10 consecutive amino acids that are coded by a nucleotide sequence that bridges the junction created by the splicing of the 3′ end of exon 11 to the 5′ end of intron 11′ or the 3′ end of intron 11′ to the 5′ end of exon 12 in the case of P2RX4sv1, by the splicing of the 3′ end of exon 9 to the 5′ end of intron 9 or the 3′ end of intron 9 to the 5′ end of exon 10 in the case of P2RX4sv2, or by the splicing of the 3′ end of exon 10 to the 5′ end of exon 11 5′ extension or the 3′ end of exon 11 5′ extension to the 5′ end of exon 11 in the case of P2RX4sv3. Similarly, the selective binding assays may also be performed using a polypeptide fragment of a purinergic receptor P2X isoform polypeptide that is not P2RX4sv1, P2RX4sv2, or P2RX4sv3, wherein the polypeptide fragment comprises at least 10 consecutive amino acids that are coded by: a nucleotide sequence that bridges the junction created by the splicing of the 3′ end of exon 11 to the 5′ end of exon 12, the splicing of the 3′ end of exon 9 to the 5′ end of exon 10, or the splicing of the 3′ end of exon 10 to the 5′ end of exon 11 of the P2RX4 gene.

P2RX3 and P2RX4 Functional Assays

P2RX3 encodes subunit 3 and P2RX4 encodes subunit 4 of a highly conserved purinergic receptor P2X ligand-gated ion channel that is implicated in neurological disorders such as chronic inflammatory pain, acute pain, neuropathic pain, visceral pain, and tactile allodynia. Splice variants of purinergic receptor P2X may exhibit different ligand gate activity and different binding affinities for compounds, peptides and other small molecules. The identification of P2RX3sv1 and P2RX3sv2 as splice variants of P2RX3 provides a means of screening for compounds that bind to P2RX3sv1 and/or P2RX3sv2 protein thereby altering the activity or regulation of P2RX3sv1 and/or P2RX3sv2 ligand-gated ion channels channels. Additionally, the identification of P2RX4sv1, P2RX4sv2, and P2RX4sv3 as splice variants of P2RX4 provides a means of screening for compounds that bind to P2RX4sv1, P2RX4sv2, and/or P2RX4sv3 protein thereby altering the activity or regulation of P2RX4sv1, P2RX4sv2, and/or P2RX4sv3 ligand-gated ion channels. Assays involving a functional P2RX3sv1, P2RX3sv2, P2RX4sv1, P2RX4sv2, or P2RX4sv3 polypeptide can be employed for different purposes, such as selecting for compounds active at P2RX3sv1, P2RX3sv2, P2RX4sv1, P2RX4sv2, or P2RX4sv3; evaluating the ability of a compound to affect the ion channel activity of each respective splice variant; and mapping the activity of different P2RX3sv1, P2RX3sv2, P2RX4sv1, P2RX4sv2, and P2RX4sv3 regions. P2RX3sv1, P2RX3sv2, P2RX4sv1, P2RX4sv2, and P2RX4sv3 activity can be measured using different techniques such as: detecting a change in the intracellular conformation of P2RX3sv1, P2RX3sv2, P2RX4sv1, P2RX4sv2, or P2RX4sv3; detecting a change in the intracellular location of P2RX3sv1, P2RX3sv2, P2RX4sv1, P2RX4sv2, or P2RX4sv3; or measuring the ion channel activity of P2RX3sv1, P2RX3sv2, P2RX4sv1, P2RX4sv2, or P2RX4sv3.

Recombinantly expressed P2RX3sv1, P2RX3sv2, P2RX4sv1, P2RX4sv2, and P2RX4sv3 can be used to facilitate the determination of whether a compound's activity in a cell is dependent upon the presence of P2RX3sv1, P2RX3sv2, P2RX4sv1, P2RX4sv2, or P2RX4sv3. For example, P2RX3sv1 can be expressed by an expression vector in a cell line and used in a co-culture growth assay, such as described in U.S. Pat. No. 6,518,035, to identify compounds that alter the growth of the cell expressing P2RX3sv1 from the expression vector as compared to the same cell line but lacking the P2RX3sv1 expression vector. Alternatively, determination of whether a compound's activity on a cell is dependent upon the presence of P2RX3sv1 can also be done using gene expression profile analysis methods as described, for example, in U.S. Pat. No. 6,324,479. Similar assays can also be used for P2RX3sv2, P2RX4sv1, P2RX4sv2, or P2RX4sv3.

Techniques for measuring ligand gated ion channel activity are well known in the art. Methods for expressing purinergic receptor P2X ion channels in Xenopus oocytes and monitoring the activity of these channels, including analyzing the effect of compounds on the activity of purinergic receptor P2X ion channel activity, have been described previously (U.S. Pat. No. 6,214,581; U.S. Pat. No. 6,242,216; Townsend-Nicholson et al., 1999, Mol. Brain Res. 64:246-254; Jarvis et al., 2002, Proc. Natl. Acad. Sci. USA 99:17179-17184). The patch clamp technique measures ion current through ion channel proteins and can be used to analyze the effect of drugs on ion channel function. The activity of these channels can be measured electrically (single channel recording) or, alternatively, the patch can be ruptured allowing measurements of the combined channel activity of the entire cell membrane (whole cell recording) (Ding and Sachs, 1999, J Gen Physiol. 113:695-720; Jarvis et al., 2002, Proc. Natl. Acad. Sci. USA 99:17179-17184; Burgard et al., 1999, J. Neurophysiol. 82:1590-1598). Other methods for measuring ion channel activity include optical reading of potential-sensitive dyes or calcium ion-sensitive fluorescent indicators (U.S. Pat. No. 6,214,581; Bianchi et al., 1999, Eur. J. Pharmacol. 376:127-138; Jarvis et al., 2002, Proc. Natl. Acad. Sci. USA 99:17179-17184). High throughput methods for assaying ion channel activity have also been described (see WO 03/006103A2; US 2002/0028480; US 2004/0132187). A variety of other assays have been used to investigate the properties of purinergic receptor P2X channels and therefore would also be applicable to the measurement of P2RX3sv1, P2RX3sv2, P2RX4sv1, P2RX4sv2, or P2RX4sv3 function.

P2RX3sv1, P2RX3sv2, P2RX4sv1, P2RX4sv2, or P2RX4sv3 functional assays can be performed using cells expressing P2RX3sv1, P2RX3sv2, P2RX4sv1, P2RX4sv2, or P2RX4sv3 at a high level. These proteins will be contacted with individual compounds or preparations containing different compounds. A preparation containing different compounds where one or more compounds affect P2RX3sv1, P2RX3sv2, P2RX4sv1, P2RX4sv2, or P2RX4sv3 in cells over-producing P2RX3sv1, P2RX3sv2, P2RX4sv1, P2RX4sv2, or P2RX4sv3 as compared to control cells containing an expression vector lacking P2RX3sv1, P2RX3sv2, P2RX4sv1, P2RX4sv2, or P2RX4sv3 coding sequences, can be divided into smaller groups of compounds to identify the compound(s) affecting P2RX3sv1, P2RX3sv2, P2RX4sv1, P2RX4sv2, or P2RX4sv3 activity, respectively.

P2RX3sv1, P2RX3sv2, P2RX4sv1, P2RX4sv2, or P2RX4sv3 functional assays can be performed using recombinantly produced P2RX3sv1, P2RX3sv2, P2RX4sv1, P2RX4sv2, or P2RX4sv3 present in different environments. Such environments include, for example, cell extracts and purified cell extracts containing the P2RX3sv1, P2RX3sv2, P2RX4sv1, P2RX4sv2, or P2RX4sv3 expressed from recombinant nucleic acid; and the use of purified P2RX3sv1, P2RX3sv2, P2RX4sv1, P2RX4sv2, or P2RX4sv3 produced by recombinant means that is introduced into a different environment suitable for measuring ion channel activity.

Modulating P2RX3sv1, P2RX3sv2, P2RX4sv1, P2RX4sv2, and P2RX4sv3 Expression

P2RX3sv1, P2RX3sv2, P2RX4sv1, P2RX4sv2, or P2RX4sv3 expression can be modulated as a means for increasing or decreasing P2RX3sv1, P2RX3sv2, P2RX4sv1, P2RX4sv2, or P2RX4sv3 activity, respectively. Such modulation includes inhibiting the activity of nucleic acids encoding the P2RX3 isoform target to reduce P2RX3 isoform protein or polypeptide expression, or supplying P2RX3 nucleic acids to increase the level of expression of the P2RX3 target polypeptide thereby increasing P2RX3 activity. Such modulation includes inhibiting the activity of nucleic acids encoding the P2RX4 isoform target to reduce P2RX4 isoform protein or polypeptide expression, or supplying P2RX4 nucleic acids to increase the level of expression of the P2RX4 target polypeptide thereby increasing P2RX4 activity.

Inhibition of P2RX3sv1, P2RX3sv2, P2RX4sv1, P2RX4sv2, and P2RX4sv3 Activity

P2RX3sv1, P2RX3sv2, P2RX4sv1, P2RX4sv2, or P2RX4sv3 nucleic acid activity can be inhibited using nucleic acids recognizing P2RX3sv1, P2RX3sv2, P2RX4sv1, P2RX4sv2, or P2RX4sv3 nucleic acid and affecting the ability of such nucleic acid to be transcribed or translated. Inhibition of P2RX3sv1, P2RX3sv2, P2RX4sv1, P2RX4sv2, or P2RX4sv3 nucleic acid activity can be used, for example, in target validation studies.

A preferred target for inhibiting P2RX3sv1, P2RX3sv2, P2RX4sv1, P2RX4sv2, or P2RX4sv3 is mRNA stability and translation. The ability of P2RX3sv1, P2RX3sv2, P2RX4sv1, P2RX4sv2, or P2RX4sv3 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify sodium channel activity have been described previously (Keller et al., 2000, J. Pharmacol. Exp. Ther. 295(1): 367-72). Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing P2RX3sv1, P2RX3sv2, P2RX4sv1, P2RX4sv2, and P2RX4sv3 Expression

Nucleic acids encoding for P2RX3sv1 or P2RX3sv2 can be used, for example, to cause an increase in P2RX3 activity or to create a test system (e.g., a transgenic animal) for screening for compounds affecting P2RX3sv1 or P2RX3sv2 expression, respectively. Nucleic acids encoding for P2RX4sv1, P2RX4sv2, or P2RX4sv3 can be used, for example, to cause an increase in P2RX4 activity or to create a test system (e.g., a transgenic animal) for screening for compounds affecting P2RX4sv1, P2RX4sv2, or P2RX4sv3 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of P2RX3sv1, P2RX3sv2, P2RX4sv1, P2RX4sv2, and P2RX4sv3 Using RT-PCR

To identify variants of the “normal” splicing of exon regions encoding P2RX3 and P2RX4, RT-PCR was used. In particular, splicing variations affecting the C-terminal cytoplasmic region of P2RX3 and P2RX4 were sought based upon information on the paralogous P2RX2 splice variants. Koshimizu et al. (1998, Mol. Endocrinol. 12:901-913) demonstrated that P2RX2 splice variants with alterations in the extracellular region were non-functional, while variants with alterations in the cytoplasmic C-terminal region retained ATP-gated ion channel activity.

RT-PCR

The structure of P2RX3 mRNA in the region corresponding to exons 9 to 12 was determined for RNA extracted from human testis and dorsal root ganglia (DRG) using an RT-PCR based assay. Total RNA isolated from human testis and DRG was obtained from BD Biosciences Clontech (Palo Alto, Calif.). RT-PCR primers were selected that were complementary to sequences in exon 9 and exon 12 of the reference exon coding sequences in P2RX3 (NM_{—}002559). Based upon the nucleotide sequence of P2RX3 mRNA, the P2RX3 exon 9 and exon 12 primer set (hereafter P2RX3₉₋₁₂ primer set) was expected to amplify a 276 base pair amplicon representing the “reference” P2RX3 mRNA region. The P2RX3 exon 9 forward primer has the sequence:

5′ TACAAAATGGAAAATGGCAGTGAGTACC 3′ [SEQ ID NO 26]; and the P2RX3 exon 12 reverse primer has the sequence: 5′ GGGGTACACTGGGTTGGTCAAAG 3′ [SEQ ID NO 27].

Twenty-five ng of total RNA from human testis and DRG was subjected to a one-step reverse transcription-PCR amplification protocol using the Qiagen, Inc. (Valencia, Calif.), One-Step RT-PCR kit, using the following cycling conditions:

50° C. for 30 minutes;

95° C. for 15 minutes;

35 cycles of:
94° C. for 30 seconds;
63.5° C. for 40 seconds;
72° C. for 50 seconds; then
72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Several hundred clones were amplified with the P2RX3₉₋₁₂ primer set and screened by size fractionation on a 2% agarose gel in order to identify clones with variable sized inserts. Candidate variant clones were then sequenced from each end (using the same primers used for RT-PCR) by Qiagen Genomics, Inc. (Bothell, Wash.).

At least two different variant clones were obtained from human testis samples using the P2RX3₉₋₁₂ primer set (data not shown). The testis sample assayed exhibited the expected amplicon size of 276 base pairs for normally spliced P2RX3 mRNA. In addition, the testis sample assayed also exhibited an amplicon of about 210 base pairs and an amplicon of about 492 base pairs. At least one different variant clone was obtained from human DRG samples using the P2RX3₉₋₁₂ primer set (data not shown). The DRG sample assay exhibited the expected amplicon size of 276 base pairs for normally spliced P2RX3 mRNA. In addition, the DRG sample assayed also exhibited an amplicon of about 210 base pairs.

Sequence analysis of the about 210 base pair amplicon amplified using the P2RX3₉₋₁₂ primer set revealed that this amplicon form results from the splicing of exon 9 of the P2RX3 hnRNA to exon 11; that is, the coding sequence of exon 10 is completely absent. This splice variant form was designated P2RX3sv1 [SEQ ID NO 6]. Sequence analysis of the about 492 base pair amplicon amplified using the P2RX3₉₋₁₂ primer set revealed that this amplicon form results from the retention of a 216 base pair portion of intron 10 of the P2RX3 hnRNA, referred to as intron 10′. This splice variant form was designated P2RX3sv2 [SEQ ID NO 8]. Thus, the RT-PCR results suggest that P2RX3 mRNA is composed of a mixed population of molecules wherein in at least two of the P2RX3 mRNA splice junctions are altered.

Additionally, the structure of P2RX4 mRNA in the region corresponding to exons 9 to 12 was determined for RNA extracted from human testis and dorsal root ganglia (DRG) using an RT-PCR based assay. Total RNA isolated from human testis and DRG was obtained from BD Biosciences Clontech (Palo Alto, Calif.). RT-PCR primers were selected that were complementary to sequences in exon 9 and exon 12 of the reference exon coding sequences in P2RX4 (NM_{—}002560). Based upon the nucleotide sequence of P2RX4 mRNA, the P2RX4 exon 9 and exon 12 primer set (hereafter P2RX4₉₋₁₂ primer set) was expected to amplify a 272 base pair amplicon representing the “reference” P2RX4 mRNA region. The P2RX4 exon 9 forward primer has the sequence: 5′ ATCCGCTTCGACATCATTGTGTTT 3′ [SEQ ID NO 28]; and the P2RX4 exon 12 reverse primer has the sequence: 5′ CTCTCTGTTCTTTGATGGGGCTGTG 3′ [SEQ ID NO 29].

Twenty-five ng of total RNA from human testis and DRG was subjected to a one-step reverse transcription-PCR amplification protocol using the Qiagen, Inc. (Valencia, Calif.), One-Step RT-PCR kit, using the cycling conditions described above.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Several hundred clones were amplified with the P2RX4₉₋₁₂ primer set and screened by size fractionation on a 2% agarose gel in order to identify clones with variable sized inserts. Candidate variant clones were then sequenced from each end (using the same primers used for RT-PCR) by Qiagen Genomics, Inc. (Bothell, Wash.).

At least three different variant clones were obtained from human testis samples using the P2RX4₉₋₁₂ primer set (data not shown). The testis sample assayed exhibited the expected amplicon size of 272 base pairs for normally spliced P2RX4 mRNA. In addition, the testis sample assayed also exhibited an amplicon of about 330 base pairs, an amplicon of about 375 base pairs and an amplicon of about 455 base pairs.

Sequence analysis of the about 330 base pair amplicon amplified using the P2RX4₉₋₁₂ primer set revealed that this amplicon form results from the retention of a 58 base pair portion of intron 11 of the P2RX4 hnRNA, referred to as intron 11′. This splice variant form was designated P2RX4sv1 [SEQ ID NO 10]. Sequence analysis of the about 375 base pair amplicon amplified using the P2RX4₉₋₁₂ primer set revealed that this amplicon form results from the retention of intron 9 of the P2RX4 hnRNA. This splice variant form was designated P2RX4sv2 [SEQ ID NO 12]. Sequence analysis of the about 455 base pair amplicon amplified using the P2RX4₉₋₁₂ primer set revealed that this amplicon form results from the extension at the 5′ end of exon 11 of the P2RX4 hnRNA with 183 base pairs from intron 10, referred to as exon 11 5′ extension. This splice variant form was designated P2RX4sv3 [SEQ ID NO 14]. Thus, the RT-PCR results suggest that P2RX4 mRNA is composed of a mixed population of molecules wherein in at least three of the P2RX4 mRNA splice junctions are altered.

### Example 2

### Cloning of P2RX3sv1, P2RX3sv2 P2RX4sv1, P2RX4sv2, and P2RX4sv3

RT-PCR and sequencing data indicate that in addition to the normal P2RX3 reference mRNA sequence, NM_{—}002559, encoding P2RX3 protein, NP_{—}002550, two novel splice variant forms of P2RX3 mRNA also exist in testis and DRG tissues.

Clones having nucleotide sequences comprising the splice variants identified in Example 1 (hereafter referred to as P2RX3sv1 or P2RX3sv2) are isolated using a 5′ “forward” P2RX3 primer and a 3′ “reverse” P2RX3 primer, to amplify and clone the entire P2RX3sv1 or P2RX3sv2 mRNA coding sequences, respectively. The same 5′ “forward” primer is designed for isolation of full length clones corresponding to the P2RX3sv1 and P2RX3sv2 splice variants and has the nucleotide sequence of 5′ ATGAACTGCATATCCGACTTCTTCACCTATGAGACCACCAAGTCG 3′ [SEQ ID NO 30]. The same 3′ “reverse” primer is designed for isolation of full length clones corresponding to the P2RX3sv1 and P2RX3sv2 splice variants and has the nucleotide sequence of 5′ CTAGTGGCCTATGGAGAAGGCCCCCGAATCGGTGGACTGCTTCTCCGC 3′ [SEQ ID NO 31].

Clones having nucleotide sequences comprising the splice variants identified in Example 1 (hereafter referred to as P2RX4sv1, P2RX4sv2, or P2RX4sv3) are isolated using a 5′ “forward” P2RX4 primer and a 3′ “reverse” P2RX4 primer, to amplify and clone the entire P2RX4sv1, P2RX4sv2, or P2RX4sv3 mRNA coding sequences, respectively. The same 5′ “forward” primer is designed for isolation of full length clones corresponding to the P2RX4sv1, P2RX4sv2, and P2RX4sv3 splice variants and has the nucleotide sequence of 5′ ATGGCGGGCTGCTGCGCCGCGCTGGCGGCCTTCCTGTTCGAGTAC 3′ [SEQ ID NO 32]. The same 3′ “reverse” primer is designed for isolation of full length clones corresponding to the P2RX4sv1, P2RX4sv2, and P2RX4sv3 splice variants and has the nucleotide sequence of 5′ TCACTGGTCCAGCTCACTAGCAAGACCCTGCTCGTAATCTTCCAC 3′ [SEQ ID NO 33].

The P2RX3sv1 and P2RX3sv2 cDNA sequences are cloned using a combination of reverse transcription (RT) and polymerase chain reaction (PCR). More specifically, about 25 ng of testis polyA mRNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the P2RX3 “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted P2RX3sv1 and P2RX3sv2 mRNAs, products of about 1128 and 1410 bases, respectively. The remainder of the 50 μl PCR reactions from testis is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

The P2RX4sv1, P2RX4sv2, and P2RX4sv3 cDNA sequences are cloned using a combination of reverse transcription (RT) and polymerase chain reaction (PCR) on testis polyA mRNA using the P2RX4 “forward” and “reverse” primers as described above. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted P2RX4sv1, P2RX4sv2, and P2RX4sv3 mRNAs, products of about 1225, 1270, and 1350 bases, respectively. The remainder of the 50 μl PCR reactions from testis is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified P2RX3sv1, P2RX3sv2, P2RX4sv1, P2RX4sv2, and P2RX4sv3 RT-PCR products from testis are used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative P2RX3sv1 clones are identified and prepared for a PCR reaction to confirm the presence of the expected P2RX3sv1 exon 9 to exon 11 splice variant structure. Twelve putative P2RX3sv2 clones are identified and prepared for a PCR reaction to confirm the presence of the expected P2RX3sv2 exon 10 to intron 10′ and intron 10′ to exon 11 splice variant structures. Twelve putative P2RX4sv1 clones are identified and prepared for a PCR reaction to confirm the presence of the expected P2RX4sv1 exon 11 to intron 11′ and intron 11′ to exon 12 splice variant structures. Twelve putative P2RX4sv2 clones are identified and prepared for a PCR reaction to confirm the presence of the expected P2RX4sv2 exon 9 to intron 9 and intron 9 to exon 10 splice variant structures. Twelve putative P2RX4sv3 clones are identified and prepared for a PCR reaction to confirm the presence of the expected P2RX4sv3 exon 10 to exon 11 5′ extension and exon 11 5′ extension to exon 11 splice variant structures. A 25 μl PCR reaction is performed as described above (RT-PCR section) to detect the presence of P2RX3sv1, except that the reaction includes miniprep DNA from the TOPO TA/P2RX3sv1 ligation as a template. An additional 25 μl PCR reaction is performed as described above (RT-PCR section) to detect the presence of P2RX3sv2, except that the reaction includes miniprep DNA from the TOPO TA/P2RX3sv2 ligation as a template. An additional 25 μl PCR reaction is performed as described above (RT-PCR section) to detect the presence of P2RX4sv1, except that the reaction includes miniprep DNA from the TOPO TA/P2RX4sv1 ligation as a template. An additional 25 μl PCR reaction is performed as described above (RT-PCR section) to detect the presence of P2RX4sv2, except that the reaction includes miniprep DNA from the TOPO TA/P2RX4sv2 ligation as a template. An additional 25 μl PCR reaction is performed as described above (RT-PCR section) to detect the presence of P2RX4sv3, except that the reaction includes miniprep DNA from the TOPO TA/P2RX4sv3 ligation as a template. About 10 μl of each 25 μl PCR reaction is run on a 1% agarose gel and the DNA bands generated by the PCR reaction are visualized and photographed on a UV light box to determine which minipreps samples have PCR product of the size predicted for the corresponding P2RX3sv1, P2RX3sv2, P2RX4sv1, P2RX4sv2, and P2RX4sv3 splice variant mRNAs. Clones having the P2RX3sv1 structure are identified based upon amplification of an amplicon band of 1128 base pairs, whereas a normal reference P2RX3 clone will give rise to an amplicon band of 1194 base pairs. DNA sequence analysis of the P2RX3sv1 cloned DNAs confirms a polynucleotide sequence representing the deletion of exon 10. Clones having the P2RX3sv2 structure are identified based upon amplification of an amplicon band of 1410 base pairs, whereas a normal reference P2RX3 clone will give rise to an amplicon band of 1194 base pairs. DNA sequence analysis of the P2RX3sv2 cloned DNAs confirm a polynucleotide sequence representing SEQ ID NO 8. Clones having the P2RX4sv1 structure are identified based upon the amplification of an amplicon band of 1225 base pairs, whereas a normal reference P2RX4 clone will give rise to an amplicon band of 1167 base pairs. DNA sequence analysis of the P2RX4sv1 cloned DNAs confirm a polynucleotide sequence representing SEQ ID NO 10. Clones having the P2RX4sv2 structure are identified based upon the amplification of an amplicon band of 1270 base pairs, whereas a normal reference P2RX4 clone will give rise to an amplicon band of 1167 base pairs. DNA sequence analysis of the P2RX4sv2 cloned DNAs confirm a polynucleotide sequence representing SEQ ID NO 12. Clones having the P2RX4sv3 structure are identified based upon the amplification of an amplicon band of 1350 base pairs, whereas a normal reference P2RX4 clone will give rise to an amplicon band of 1167 base pairs. DNA sequence analysis of the P2RX4sv3 cloned DNAs confirm a polynucleotide sequence representing SEQ ID NO 14.

Summary of the P2RX3sv1, P2RX3sv2, P2RX4sv1, P2RX4sv2, and P2RX4sv3 Polynucleotides

The polynucleotide sequence of P2RX3sv1 mRNA [SEQ ID NO 6] contains an open reading frame that encodes a P2RX3sv1 protein [SEQ ID NO 7] similar to the reference P2RX3 protein (NP_{—}002550), but lacking amino acids encoded by exon 10 of the full length coding sequence of reference P2RX3 mRNA (NM_{—}002559). The alternative splicing of exon 9 to exon 11 deletes a 66 base pair region corresponding to exon 10, but the protein reading frame at the novel exon 9/exon 11 splice junction is maintained in the same reading frame as that used to encode the reference P2RX3 protein (NP_{—}002550). Therefore the P2RX3sv1 protein is missing an internal 22 amino acid region that corresponds to the amino acids encoded by exon 10 as compared to the reference P2RX3 (NP_{—}002550).

The polynucleotide sequence of P2RX3sv2 mRNA [SEQ ID NO 8] contains an open reading frame that encodes a P2RX3sv2 protein [SEQ ID NO 9] similar to the reference P2RX3 protein (NP_{—}002550), but retaining amino acids encoded by a 216 base pair region corresponding to a portion of intron 10, referred to as intron 10′, of the full length coding sequence of the reference P2RX3 mRNA (NM_{—}002559). The insertion of the 216 base pair region does not change the protein translation reading frame in comparison to the reference P2RX3 protein reading frame. The insertion of the 216 base pair region does create a premature termination codon 121 nucleotides downstream of the exon 10/intron 10′ splice junction. Therefore, the P2RX3sv1 protein has a unique 40 amino acid region at the C-terminus and is also lacking the amino acids encoded by the nucleotides downstream of the premature stop codon as compared to the reference P2RX3 protein (NP_{—}002550).

The polynucleotide sequence of P2RX4sv1 mRNA [SEQ ID NO 10] contains an open reading frame that encodes a P2RX4sv1 protein [SEQ ID NO 11] similar to the reference P2RX4 protein (NP_{—}002551), but retaining amino acids encoded by a 58 base pair region corresponding to a portion of intron 11, referred to as intron 11′, of the full length coding sequence of the reference P2RX4 mRNA (NM_{—}002560). The insertion of the 58 base pair region does change the protein translation reading frame in comparison to the reference P2RX4 protein reading frame, creating a carboxy terminal peptide region that is unique to P2RX4sv1. The insertion also creates a premature termination codon 16 nucleotides downstream of the exon 11/intron 11′ splice junction. Therefore, the P2RX4sv1 protein has a unique 5 amino acid region at the C-terminus and is also lacking the amino acids encoded by the nucleotides downstream of the premature stop codon as compared to the reference P2RX4 protein (NP_{—}002551).

The polynucleotide sequence of P2RX4sv2 mRNA [SEQ ID NO 12] contains an open reading frame that encodes a P2RX4sv2 protein [SEQ ID NO 13] similar to the reference P2RX4 protein (NP_{—}002551), but retaining amino acids encoded by the 103 base pair region corresponding to intron 9 of the full length coding sequence of the reference P2RX4 mRNA (NM_{—}002560). The insertion of the 103 base pair region does change the protein translation reading frame in comparison to the reference P2RX4 protein reading frame creating a carboxy terminal peptide region that is unique to P2RX4sv2. The frameshift also creates a premature termination codon 12 nucleotides downstream of the intron 9/exon 10 splice junction. Therefore, the P2RX4sv2 protein has a unique 38 amino acid region at the C-terminus and is also lacking the amino acids encoded by the nucleotides downstream of the premature stop codon as compared to the reference P2RX4 protein (NP_{—}002551).

The polynucleotide sequence of P2RX4sv3 mRNA [SEQ ID NO 14] contains an open reading frame that encodes a P2RX4sv3 protein [SEQ ID NO 15] similar to the reference P2RX4 protein (NP_{—}002551), but retaining amino acids encoded by the a 183 base pair region corresponding to a portion of intron 10, referred to as exon 11 5′ extension of the full length coding sequence of the reference P2RX4 mRNA (NM_{—}002560). The insertion of the 183 base pair region does not change the protein translation reading frame in comparison to the reference P2RX4 protein reading frame. The insertion of the 183 base pair region does create a premature termination codon 175 nucleotides downstream of the exon 10/exon 11 5′ extension splice junction. Therefore, the P2RX4sv3 protein has a unique 58 amino acid region at the C-terminus and is also lacking the amino acids encoded by the nucleotides downstream of the premature stop codon as compared to the reference P2RX4 protein (NP_{—}002551).

### Example 3

### Analyzing Ion Channel Activity of P2RX3, P2RX4, and Other Purinergic Receptor P2X Isoforms

The preparation of Xenopus laevis oocytes, injection with receptor RNA or DNA, and measurement of receptor responses using two-electrode voltage-clamp follows procedures described by Briggs et al. (1995, Neuropharmacol. 34:583-590). Ooctyes are maintained at 17-18° C. in normal Barth's solution (90 mM NaCl, 1 mM KCl, 0.66 mM NaNO₃, 0.74 mM CaCl₂, 0.82 mM MgCl₂, 2.4 mM NaHCO₃, 2.4 mM sodium pyruvate, and 10 mM NaN-(2-hydroxy-ethyl)-piperazine-N′-(2-ethanesulfonic acid) (“HEPES”) buffer, final pH 7.55) containing 100 μg/ml gentamicin. Responses are measured at a holding potential of −60 mV in modified Barth's solution containing 10 mM BaCl₂ and lacking CaCl₂ and MgCl₂ (final pH 7.4). However, in some experiments, the cell potential is intentionally varied in order to determine the response current-voltage relationship. Agonist is applied briefly using a computer-controlled solenoid valve and a push/pull applicator positioned to within 200-400 μm from the oocyte. Responses are recorded by the computer in synchrony with agonist application. Antagonists are included with agonist in the push/pull applicator and are applied to the bath by superfusion for at least 3 minutes before application of agonist. Responses are quantified by measuring the peak amplitude.

DNA for injection into the oocytes is prepared from the the appropriate P2RX3 or P2RX4 isoform cloned into standard expression vectors, such as pCDNA3.1 (Invitrogen). The clone is grown up and prepared in large scale using the QIAgen maxiprep DNA preparation system according to manufacturer's instructions. The DNA is ethanol precipitated and resuspended in TE buffer. For RNA production, the purinergic receptor P2X isoform-pCDNA3.1 construct is linearized by digestion with the restriction enzyme, and RNA is transcribed using the T7 RNA polymerase and the mMessage mMachine in vitro translation kit from Ambion according to the manufacturer's instructions.

For functional analysis of human P2RX3 or P2RX4 receptors, 10 ng of human receptor DNA prepared as described above are injected into the nucleus of Xenopus oocytes. Oocytes are incubated in normal Barth's solution containing 100 μg/ml gentamicin for 2-7 days following injection. The response to 10 μM ATP is then recorded. Alternatively, 50 ng of receptor RNA prepared as described above is injected into the oocyte cytoplasm.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed is:

1. A purified human nucleic acid comprising SEQ ID NO 6, or the complement thereof.

2. The purified nucleic acid of claim 1, wherein said nucleic acid comprises a region encoding SEQ ID NO 7.

3. The purified nucleic acid of claim 1, wherein said nucleotide sequence encodes a polypeptide consisting of SEQ ID NO 7.

4. A purified polypeptide comprising SEQ ID NO 7.

5. The polypeptide of claim 4, wherein said polypeptide consists of SEQ ID NO 7.

6. An expression vector comprising a nucleotide sequence encoding SEQ ID NO 7, wherein said nucleotide sequence is transcriptionally coupled to an exogenous promoter.

7. The expression vector of claim 6, wherein said nucleotide sequence encodes a polypeptide consisting of SEQ ID NO 7.

8. The expression vector of claim 6, wherein said nucleotide sequence comprises SEQ ID NO 6.

9. The expression vector of claim 6, wherein said nucleotide sequence consists of SEQ ID NO 6.

10. A method of screening for compounds able to bind selectively to P2RX3sv1 comprising the steps of:
(a) providing a P2RX3sv1 polypeptide comprising SEQ ID NO 7;
(b) providing one or more purinergic receptor P2X isoform polypeptides that are not P2RX3sv1;
(c) contacting said P2RX3sv1 polypeptide and said purinergic receptor P2X isoform polypeptide that is not P2RX3sv1 with a test preparation comprising one or more compounds; and
(d) determining the binding of said test preparation to said P2RX3sv1 polypeptide and to said purinergic receptor P2X isoform polypeptide that is not P2RX3sv1, wherein a test preparation which binds to said P2RX3sv1 polypeptide, but does not bind to said purinergic receptor P2X isoform polypeptide that is not P2RX3sv1, contains a compound that selectively binds said P2RX3sv1 polypeptide.

11. The method of claim 10, wherein said P2RX3sv1 polypeptide is obtained by expression of said polypeptide from an expression vector comprising a polynucleotide encoding SEQ ID NO 7.

12. The method of claim 11, wherein said polypeptide consists of SEQ ID NO 7.

13. A method for screening for a compound able to bind to or interact with a P2RX3sv1 protein or a fragment thereof comprising the steps of:
(a) expressing a P2RX3sv1 polypeptide comprising SEQ ID NO 7 or fragment thereof from a recombinant nucleic acid;
(b) providing to said polypeptide a labeled P2RX3 ligand that binds to said polypeptide and a test preparation comprising one or more compounds; and
(c) measuring the effect of said test preparation on binding of said labeled P2RX3 ligand to said polypeptide, wherein a test preparation that alters the binding of said labeled P2RX3 ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said P2RX3sv1 ligand is a P2RX3 inhibitor.

18. The method of claim 16, wherein said expression vector comprises SEQ ID NO 6 or a fragment of SEQ ID NO 6.

19. The method of claim 16, wherein said polypeptide comprises SEQ ID NO 7 or a fragment of SEQ ID NO 7.

20. A method of screening for P2RX3sv1 activity comprising the steps of:
(a) contacting a cell expressing a recombinant nucleic acid encoding P2RX3sv1 comprising SEQ ID NO 7 with a test preparation comprising one or more test compounds; and
(b) measuring the effect of said test preparation on ion channel activity.
